(19) United States
(12) Patent Application Publication
(10) Pub. No.: US 2021/0322427 A1
(43) Pub. Date: Oct. 21, 2021

(54) INHIBITION OF RIP KINASES FOR TREATING NEURODEGENERATIVE DISORDERS

(71) Applicant: The Johns Hopkins University, Baltimore, MD (US)

(72) Inventors: Seulki LEE, Ellicott City, MD (US); Han Seok KO, Lutherville, MD (US); Ted M. DAWSON, Baltimore, MD (US); Martin G. POMPER, Baltimore, MD (US); Donghoon KIM, Seoul (KR); Yumin OH, Elkridge, MD (US); Seung-Hwan KWON, Ellicott City, MD (US); Yong Joo PARK, Suwon-si, Gyeonggi-do (KR)

(21) Appl. No.: 17/271,966

(22) PCT Filed: Aug. 30, 2019
(86) PCT No.: PCT/US2019/049071

§ 371 Date: Feb. 26, 2021

Related U.S. Application Data
(60) Provisional application No. 62/725,647, filed on Aug. 31, 2018.

(51) Int. Cl. A61K 31/5377; A61K 31/44; A61K 31/5025; A61K 31/4439; A61K 31/529; A61K 31/504; A61K 31/519; A61K 31/4709; A61P 25/28

(52) U.S. Cl. CPC A61K 31/5377; A61K 31/44; A61K 31/5025; A61K 31/4439; A61P 25/28; A61K 31/504; A61K 31/519; A61K 31/4709; A61K 31/529

(57) ABSTRACT

Provided herein are compositions comprising a RIPK2 inhibitor and methods of using the RIPK2 inhibitor for treating or preventing neurodegenerative diseases or disorders. Also provided herein are methods of screening or identifying therapeutic agents useful for treating or preventing neurodegenerative diseases or disorders.

### STATEMENT REGARDING FEDERALLY-SPONSORED RESEARCH AND DEVELOPMENT

The U.S. government has a paid-up license in this invention and the right in limited circumstances to require the patent owner to license others on reasonable terms as provided for by the terms of R01NS107404 awarded by the National Institutes of Health.

Part of the work performed during development of this invention utilized U.S. Government funds. The U.S. Government has certain rights in this invention.

### FIELD OF THE INVENTION

Embodiments of the invention are directed to Receptor-Interacting Protein (RIP) kinases for the prevention and treatment of neurodegenerative diseases.

### BACKGROUND

The nervous system is divided into two parts: the central nervous system (CNS), which includes the brain and the spinal cord, and the peripheral nervous system, which includes nerves and ganglions outside of the brain and the spinal cord. While the peripheral nervous system is capable of repair and regeneration, the CNS is unable to self-repair and regenerate.

Neurodegeneration refers to the progressive loss of function or structure of neurons. Neurodegenerative diseases, such as Alzheimer's disease (AD), Parkinson's disease (PD), amyotrophic lateral sclerosis (ALS), multiple sclerosis (MS), dementia, and Huntington's disease are the results of neurodegenerative processes and affect millions of people worldwide. These age-related insults to the CNS cause progressive deterioration of neuronal structures and functions, axonal loss, disrupt neuronal connections, and ultimately result in permanent blindness, paralysis, and other losses in cognitive, motor, and sensory functions. Treatment options are currently very limited.

### SUMMARY OF THE INVENTION

In various embodiments, the present invention is based, at least in part, upon the development and use of RIPK2 inhibitors with neuroprotective and disease modifying effects on the central nervous system.

Embodiments of the invention are directed, inter alia, to compositions for the prevention and treatment of neurodegenerative diseases or disorders by inhibiting RIP kinase 2 (RIPK2) and optionally other RIP kinases. Embodiments of the invention are also directed to methods of treatment of neurodegenerative diseases or disorders comprising administering to a subject at least one RIPK2 inhibitor.

In certain embodiments, the present invention provides a method of preventing or treating a neurodegenerative disease or disorder. In some embodiments, the method comprises administering to a subject in need thereof, a therapeutically effective amount of at least one RIPK2 inhibitor or a pharmaceutical composition comprising at least one RIPK2 inhibitor.

In certain embodiments, the at least one RIPK2 inhibitor inhibits activity and/or expression of RIPK2. In some embodiments, the RIPK2 inhibitor is selective over other RIP kinases such as RIPK1 and/or RIPK3, e.g., with a selectivity of about 2-fold, about 3-fold, about 4-fold, about 5-fold, about 10-fold, or higher. In some embodiments, the RIPK2 inhibitor has substantially no activity against other RIP kinases. However, in some embodiments, the RIPK2 inhibitor can also be a dual or multi RIP kinases inhibitor, or a pan-RIP kinase inhibitor.

In some embodiments, the present disclosure is based, at least in part, upon the identification of compositions and methods for blocking or reversing microglia activation and reactive astrocyte formation, which are key cells involved in the progression of neurodegenerative diseases, to halt triggering of a cascade of neuroinflammation and neurotoxic pathways. Accordingly, in some embodiments, the disclosure provides a method of protecting neuronal cells by blocking gliosis (activation of microglia and/or astrocytes) and releasing toxic molecules from activated microglia and/or reactive astrocytes through targeting overexpressed and phosphorylated RIPK2 in the brain.

Various RIPK2 inhibitors are suitable for the compositions and methods herein. In certain embodiments, the RIPK2 inhibitor can comprise small molecules, siRNAs, shRNAs, micro RNAs, antibodies, aptamers, DNAzymse, enzymes, a gene editing system, hormones, inorganic compounds, oligonucleotides, organic compounds, polynucleotides, peptides, ribozymes, or synthetic compounds.

In certain embodiments, the RIPK2 inhibitor is a polynucleotide molecule. According to certain embodiments, the polynucleotide molecule is a nucleic acid sequence or a molecule capable of hybridizing to nucleic acids encoding or controlling RIPK2 expression. Exemplary nucleic acid sequences suitable in the context of the present invention include, but are not limited to, an RNA inhibiting (RNAi) molecule, an antisense molecule, and a ribozyme. Each possibility represents a separate embodiment of the invention. As used herein, the term RNAi describes a short RNA sequence capable of regulating the expression of target genes by binding to complementary sites in the target gene transcripts to cause translational repression or transcript degradation.

In some embodiments, the RIPK2 gene expression is down-regulated by at least 25%, at least 50%, at least 70%, at least 80%, or at least 90% as compared to an appropriate control. In certain other embodiments, partial down-regulation is preferred. Examples for expression-inhibiting (down-regulating or silencing) oligonucleotides are antisense molecules, RNA interfering molecules (RNAi), and enzymatic nucleic acid molecules, as detailed herein.

In certain embodiments, the RIPK2 inhibitor is a small molecule capable of inhibiting the activity of RIPK2 protein. Any small molecule known to have such activity can be used according to the teachings of the present invention. According to further typical embodiments, the small molecule can be formulated within a pharmaceutical composition. According to certain embodiments, the small molecule is capable of passing through the blood brain barrier (BBB) or is formulated to pass through the BBB. There are several means for delivering compounds through the BBB as disclosed, for example, in U.S. Pat. Nos. 8,629,114, 8,497,246, and 7,981,864. For example, the RIPK2 inhibitor compounds can be fused or conjugated to BBB transfer compounds as described in the art.

In certain embodiments, the RIPK2 inhibitor selectively inhibits one or more of the following activities: NOD1-dependent activation of NFκB, NOD2-dependent activation of NF-kB, amyloid-β aggregates-induced microglial activation, alpha-synuclein aggregates-induced microglial activation, and/or A1 astrocyte formation.

In certain embodiments, by inhibiting RIPK2 activities, the levels of TNF-α, IL-1α, IL-10, IL-6, C1q, and/or activated microglia and reactive astrocytes in the brain are reduced, maintained, or restored to normal levels in the subject, as compared to an appropriate control.

In certain embodiments, by inhibiting RIPK2 activities, the levels of abnormal deposits of the brain protein such as α-synuclein (Lewy body), amyloid plaques, and/or tau are reduced, maintained at, or resorted to normal levels in the subject, as compared to an appropriate control.

In certain embodiments, by inhibiting RIPK2 activities, the treatment alleviates or restores motor deficit, improves memory functions, and/or increases the lifespan in the subject, as compared to an appropriate control.

In certain embodiments, the method herein further comprises administering to the subject an effective amount of at least one additional therapeutically active compound, e.g., additional anti-Parkinson's disease or anti-Alzheimer's disease agents. In some embodiments, the additional therapeutically active compounds can also be inhibitors of other RIP kinases, such as RIPK1, RIPK3, RIPK4, or RIPK5. However, in some embodiments, the RIPK2 inhibitor can also be the only active compound administered to the subject for the respective diseases or disorders.

In various embodiments, the RIPK2 inhibitor and/or additional therapeutically active compound is/are administered intravenously, subcutaneously, intra-arterially, intraperitoneally, ophthalmically, intramuscularly, buccally, rectally, vaginally, intraorbitally, intracerebrally, intradermally, intracranially, intraspinally, intraventricularly, intrathecally, intracisternally, intracapsularly, intrapulmonary, intranasally, transmucosally, transdermally, inhalation, or any combinations thereof. In certain embodiments, the RIPK2 inhibitor is administered orally or parenterally.

Various neurodegenerative diseases or disorders are suitable to be treated by the methods herein. In certain embodiments, the neurodegenerative disease or disorder can comprise Alzheimer's disease, amyotropic lateral sclerosis (ALS/Lou Gehrig's Disease), Parkinson's disease, multiple sclerosis, diabetic neuropathy, polyglutamine (polyQ) diseases, stroke, Fahr disease, Menke's disease, Wilson's disease, cerebral ischemia, a prion disorder, dementia, corticobasal degeneration, progressive supranuclear palsy, multiple system atrophy, hereditary spastic paraparesis, spinocerebellar atrophies, brain injury, or spinal cord injury.

In certain embodiments, the present disclosure also provides a method of identifying a therapeutic agent for a neurodegenerative disease or disorder. In some embodiments, the method comprises contacting a cell or tissue expressing RIPK2 with a candidate therapeutic agent; assaying for RIPK2 activity or expression; and measuring inhibition of RIPK2 expression or activity as compared to a control. In some embodiments, the method comprises contacting a CNS resident innate immune cell (e.g., microglia and/or astrocytes) with an agent that induces the activation of the immune cell (e.g., an abnormally aggregated protein) in the presence of a candidate therapeutic agent; measuring activation of the CNS resident innate immune cell in the presence of the candidate therapeutic agent; and identifying a therapeutic agent that inhibits activation of the CNS resident innate immune cell compared to a control. In some embodiments, the candidate therapeutic agent is a RIPK2 inhibitor.

In certain embodiments, the present disclosure provides a pharmaceutical composition comprising a therapeutically effective amount of one or more RIPK2 inhibitors as described herein.

In other embodiments, the present disclosure provides a kit for the treatment of a neurodegenerative disease or disorder. In some embodiments, the kit comprises a pharmaceutical composition comprising at least one RIPK2 inhibitor and a pharmaceutically acceptable carrier, excipient or diluent. In certain embodiments, the kit further comprises at least one additional therapeutically active compound (e.g., described herein).

Other aspects are described infra.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

FIGS. 1A-1H present graphs and pictures related to RIPK2 expression in human PD postmortem tissues. FIG. 1A presents pictures showing microglial activation in PD postmortem tissues. FIG. 1B presents pictures showing p-RIPK2 activation in PD postmortem tissues. The p-RIPK2 positive signals were quantified and represented as a bar graph in FIG. 1B. FIG. 1C shows representative confocal images with anti-p-RIPK2 (green) and the microglia marker anti-cd-11b (red). FIG. 1D presents bar graphs showing the mRNA expression levels of Nod2 and Ripk2 in the SNpc region of human postmortem tissues. FIG. 1E shows NOD2, p-RIPK2, and RIPK2 expression levels in the SNpc of human postmortem assessed by western blotting. NOD2 expression levels were quantified and represented as a bar graph in FIG. 1F. p-RIPK2 and RIPK2 expression levels were quantified and represented as a bar graph in FIG. 1G. FIG. 1H presents pictures showing results of proximity ligation assay, which shows the interaction between NOD2 and α-synuclein aggregates in the SNpc of human PD postmortem tissues.

FIG. 2 presents bar graphs showing the expression of RIPK2, NOD1 and NOD2 of mouse primary microglia activated with α-synuclein PFFs for 3 hours. The gene expression of RIPK2, NOD1 and NOD2 was measured by real-time PCR.

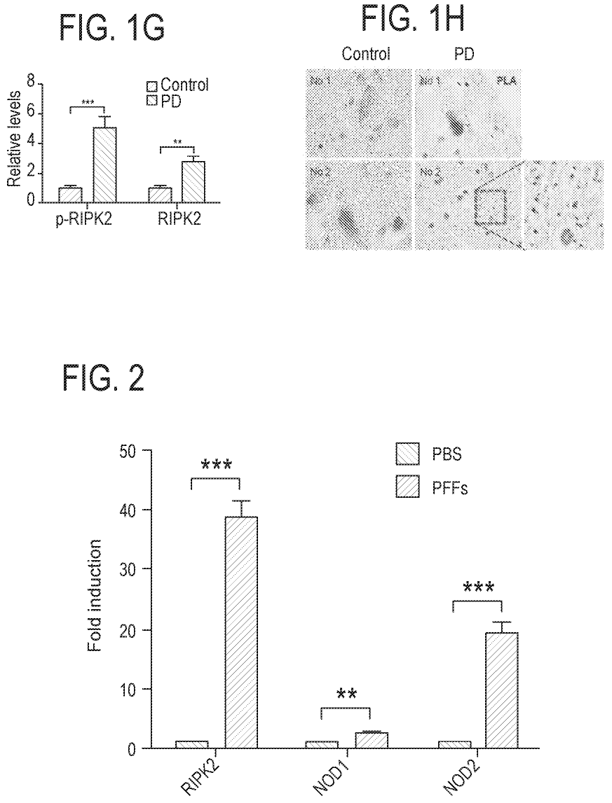

FIGS. 3A-3C are bar graphs showing the mRNA levels of A1 reactive astrocyte inducing factors such as C1q, TNFα, and IL-1α measured in PFFs-induced microglia using real-time RT-PCR. FIG. 3D shows the levels of PAN-reactive, A1-specific, and A2-specific transcripts measured in primary cultured astrocytes at 24 hours after treatment of microglia conditional medium (MCM) purified from PFFs induced WT, NOD2−/−, and RIPK2−/− primary cultured microglia. FIGS. 3E and 3F are bar graphs showing the cytotoxicity of MCM-activated astrocyte conditional medium (ACM) treated primary cultured mouse cortical neurons measured using AlamarBlue and LDH assays. The values are the mean±S.E.M. of three independent experiments (*P<0.05, **P<0.01, ***P<0.001).

FIGS. 4A and 4B present micrographs and bar graph showing morphological correlates of primary cultured microglia from wild-type (WT), NOD2 knockout (NOD2^{-/-}), and RIPK2 knockout (RIPK2^{-/-}) mice after 12 hrs of α-synuclein PFFs treatment (n=3, each group). FIGS. 4C, 4D, and 4E present bar graphs showing the mRNA expression of IL-1beta, iNOS, and chemokine Cxcl1 measured using real-time RT-PCR. FIG. 4F shows a schematic diagram of the migration assay. Primary cultured microglia were plated in upper chamber and bottom of culture dish. FIG. 4G present images showing results after 12 hours α-synuclein PFFs treatments, with the migrated cells on the bottom side of chamber stained with Iba-1 antibody. FIG. 4H presents bar graphs showing the migration index calculated through the ratio between the number of Iba-1 positive PFFs-induced migrated microglia with respect to PBS controls (n=3, each group). The values are the mean±S.E.M. of three independent experiments (*P<0.05, **P<0.01, ***P<0.001).

FIGS. 5A-5C present bar graphs showing the mRNA levels of A1 reactive astrocyte inducing factors such as C1q, TNFα, and IL-1α measured in PFFs-induced microglia using real-time RT-PCR. FIG. 5D shows the levels of PAN-reactive, A1-specific, and A2-specific transcripts measured in primary cultured astrocytes at 24 hours after treatment of α-synuclein PFFs-activated microglia conditional medium (MCM) purified from PFFs induced primary microglia with RIPK2 inhibitors Gefitinib and GSK583. FIGS. 5E and 5F present bar graphs showing the cytotoxicity of MCM-activated ACM treated primary cultured mouse cortical neurons measured using AlamarBlue and LDH assays. The values are the mean±S.E.M. of three independent experiments (*P<0.05, **P<0.01, ***P<0.001).

FIGS. 6A and 6B present pictures and bar graphs showing the ventral midbrain tissues of PFFs injected wild-type (WT), NOD2 knockout (NOD2^{-/-}), and RIPK2 knockout (RIPK2^{-/-}) mice, stained with pS129-α-synuclein or anti-Iba-1 antibodies and quantified.

FIGS. 7A-7C present bar graphs showing mRNA levels of A1 reactive astrocyte inducing factor such as C1q, TNFα, and IL-1α measured using purified microglia from WT, RIPK2 knockout and NOD2 knockout mice by immune-panning method. The mRNA levels were measured by real-time RT-PCR and represented as a bar graph. FIG. 7D shows the mRNA levels of PAN-reactive, A1-specific, and A2-specific transcripts measured in purified astrocyte from ventral midbrain area by immune-panning method. FIG. 7E shows representative immunoblots of Iba-1, GFAP, and β-actin in the ventral midbrain. FIGS. 7F and 7G present bar graphs showing quantification of Iba-1, GFAP protein levels normalized to β-actin. Error bars represent the mean±S.E.M, n=4 mice per groups. One-way ANOVA was used for statistical analysis followed by post-hoc Bonferroni test for multiple group comparison. *P<0.05, ***P<0.001 vs. PBS stereotaxic injected mice with vehicle or α-synuclein PFF stereotaxic injected mice with vehicle. n.s.: not significant.

FIG. 8A shows a representative photomicrograph of striatal sections stained for TH immunoreactivity. High power view of TH fiber density in the striatum (lower panels). The scale bars represent 100 μm (upper panels) and 50 μm (lower panels) respectively. FIG. 8B presents a bar graph showing quantification of dopaminergic fiber densities in the striatum by using Image J software. FIG. 8C shows representative photomicrographs from coronal mesencephalon sections containing TH positive neurons in PBS and α-synuclein PFF intra-striatal injected mice using stereotaxic instrument. The scale bar represents 500 μm. FIG. 8D shows representative immunoblots of TH, DAT, and β-actin in the ventral midbrain. FIG. 8E presents bar graphs showing stereology counts of TH and FIG. 8E presents bar graphs showing Nissl-positive neurons in the SNpc region. Unbiased sterologic counting was performed in the SNpc region. Error bars represent the mean±S.E.M, n=5 mice per groups. FIGS. 8G and 8H present bar graphs showing quantification of TH, and DAT protein levels normalized to β-actin. Error bars represent the mean±S.E.M, n=4 mice per groups. At six months after PBS or α-syn PFF stereotaxic intra-striatal injection, behavioral tests were performed. Results of mice on the pole (FIG. 8I) and grip strength (FIG. 8J) tests. Error bars represent the mean±S.E.M (n=12-16). One-way ANOVA was used for statistical analysis followed by post-hoc Bonferroni test for multiple group comparison. **P<0.01, ***P<0.001 vs. PBS stereotaxic injected mice with vehicle or α-syn PFF stereotaxic injected mice with vehicle. Maximum time to climb down the pole was limited to 60 sec.

FIGS. 9A and 9B show images of the ventral midbrain tissues of PFFs injected animals with RIPK2 inhibitor Gefitinib, stained with pS129-α-synuclein or anti-Iba-1 antibodies and quantified.

FIG. 10A shows p-RIPK2 expression assessed in the human hippocampus region of AD postmortem by immunohistochemistry with anti-p-RIPK2 antibody (arrowhead indicates p-RIPK2 positive signals). FIG. 10B present bar graphs showing the densities of p-RIPK2 signals in the CA1 area of hippocampus measured by ImageJ (n=3, each group).

FIG. 11 shows a representative western blot demonstrating the expression p-RIP2K and binding of NOD2 in Aβ-activated BV-2 microglia cells.

FIG. 12A shows behavioral experimental procedures. Mice were injected with AβO₁₋₄₂ (total 5 μmol, bilateral i.c.v.) and then subjected to Morris water maze test (MWMT). FIGS. 12B and 12C present bar graphs showing the data of escape latency time and probe trial session in the Morris water maze test, respectively. FIGS. 12D and 12E present bar graphs showing data of total distanced travelled and swimming speed in probe trial sessions of the MWMT, respectively. Probe trial sessions were performed for 60 sec. FIG. 12F shows representative swimming paths of mice from each group in the MWMT on the probe trial day 5. The mice were then given two trial sessions each day for four consecutive days, with an inter-trial interval of 15 min, and the escape latencies were recorded. This parameter was averaged for each session of trials and for each mouse. Error bars represent the mean±S.E.M. All behavior tests were analyzed by one-way ANOVA followed by post-hoc Bonferroni test for multiple group comparison. n=9-13 per group. *P<0.05, **P<0.01, and ***P<0.001 vs. PBS stereotaxic injected mice with vehicle or AβO₁₋₄₂ stereotaxic i.c.v. injected mice with vehicle. n.s.: not significant.

### DETAILED DESCRIPTION

### Definitions

The terminology used herein is for the purpose of describing particular embodiments only and is not intended to be limiting of the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the phrase “administration” of a compound, “administering” a compound, or other variants thereof means providing the compound or a prodrug of the compound to the subject in need of treatment.

By “antisense oligonucleotides” or “antisense compound” is meant an RNA or DNA molecule that binds to another RNA or DNA (target RNA, DNA). For example, if it is an RNA oligonucleotide, it binds to another RNA target by means of RNA-RNA interactions and alters the activity of the target RNA. An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, short, hairpin RNA (shRNA), therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds can be introduced in the form of single-stranded, double-stranded, partially single-stranded, or circular oligomeric compounds.

An antisense compound is “specifically hybridizable” when binding of the compound to the target nucleic acid interferes with the normal function of the target nucleic acid to cause a modulation of function and/or activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

Active agents that are co-administered can be concurrently or sequentially administered to an individual.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “control” refers to any reference standard suitable to provide a comparison to the expression products in the test sample. In some embodiments, the control comprises obtaining a “control sample” from which expression product levels are detected and compared to the expression product levels from the test sample. Such a control sample can comprise any suitable sample, including but not limited to a sample from a control patient with a specific neurodegenerative disease or disorder (can be stored sample or previous sample measurement) with a known outcome; normal tissue or cells isolated from a subject, such as a normal patient or the patient with a specific neurodegenerative disease or disorder, cultured primary cells/tissues isolated from a subject such as a normal subject or the patient with a specific neurodegenerative disease or disorder, adjacent normal cells/tissues obtained from the same organ or body location of the patient with a specific neurodegenerative disease or disorder, a tissue or cell sample isolated from a normal subject, or a primary cells/tissues obtained from a depository. In other embodiments, the control can comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care therapy for patients with specific neurodegenerative diseases or disorders). It will be understood by those of skill in the art that such control samples and reference standard expression product levels can be used in combination as controls in the methods of the present invention. In some embodiments, the control can comprise normal cell/tissue sample. In other embodiments, the control can comprise an expression level for a set of patients, such as a set of patients with specific neurodegenerative diseases or disorders, or for a set of patients with specific neurodegenerative diseases or disorders receiving a certain treatment, or for a set of patients with one outcome versus another outcome. In the former case, the specific expression product level, e.g., RIPK2 expression, of each patient can be assigned to a percentile level of expression, or expressed as either higher or lower than the mean or average of the reference standard expression level. In other embodiments, the control can comprise normal cells or cells from patients treated with inhibitors of RIP kinases, etc. In other embodiments, the control can also comprise a measured value for example, average level of expression of a RIP kinase gene in a population compared to the level of expression of a housekeeping gene in the same population. Such a population can comprise normal subjects, patients with specific neurodegenerative diseases or disorders who have not undergone any treatment (i.e., treatment naïve), or patients with specific neurodegenerative diseases or disorders undergoing standard of care therapy. In other embodiments, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In some embodiments, the control comprises a control sample which is of the same lineage and/or type as the test sample. In other embodiments, the control can comprise expression product levels grouped as percentiles within or based on a set of patient samples, such as all patients with specific neurodegenerative diseases or disorders. In some embodiments, a control expression product level is established wherein higher or lower levels of expression product relative to, for instance, a particular percentile, are used as the basis for predicting outcome. In other embodiments, a control expression product level is established using expression product levels from control patients with specific neurodegenerative diseases or disorders with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

As used herein, an “effective amount,” “therapeutically effective amount,” or “effective dose” is an amount of a composition (e.g., a therapeutic composition or agent) that produces at least one desired therapeutic effect in a subject, such as preventing or treating a target condition or beneficially alleviating a symptom associated with the condition.

“Mammal” covers warm blooded mammals that are typically under medical care (e.g., humans and nonhumans, such as domesticated animals). Examples include feline, canine, equine, bovine, and humans.

In the context of this invention, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, 0 such as Watson-Crick type of base pairing, Hoogsteen or reverse Hoogsteen types of base pairing, or the like.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

The terms “patient,” “subject,” and “individual” can be used interchangeably and refer to either a human or a nonhuman animal. These terms include mammals such as humans, primates, livestock animals (e.g., bovines, porcines), companion animals (e.g., canines, felines) and rodents (e.g., mice and rats).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. shRNAs can be substrates for the enzyme Dicer, and the products of Dicer cleavage can participate in RNAi. shRNAs can be derived from transcription of an endogenous gene encoding a shRNA, or can be derived from transcription of an exogenous gene introduced into a cell or organism on a vector, e.g., a plasmid vector or a viral vector. An exogenous gene encoding a shRNA can additionally be introduced into a cell or organism using other methods known in the art, e.g., lipofection, nucleofection, etc.

A “therapeutic” treatment is a treatment administered to a subject who exhibits signs of pathology, for the purpose of diminishing or eliminating those signs.

As used herein, the terms “treat,” “treating,” “treatment,” and the like refer to eliminating, reducing, or ameliorating a disease or condition, and/or symptoms associated therewith, such as reducing the frequency with which a symptom of the disease or disorder is experienced by a patient. Although not precluded, treating a disease or condition does not require that the disease, condition, or symptoms associated therewith be completely eliminated. As used herein, the terms “treat,” “treating,” “treatment,” and the like can include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein, e.g., a RIPK2 inhibitor described herein, to a subject in need of such treatment.

The term “inhibition”, “inhibiting”, “inhibit,” or “inhibitor” refer to the ability of a compound to reduce, slow, halt or prevent activity of a particular biological process (e.g., activity of RIPK2 relative to vehicle control).

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human.

### RIPK2

Microglia are the resident macrophages of the central nervous system (CNS). In response to systemic inflammation or neurodegeneration, microglia become an activated state, often referred to as M1-like proinflammatory state, and chronic activation of microglia can potentially causes neurotoxicity and facilitate neurodegenerative disease progression. Activation of microglia leads to the conversion of resting astrocytes to reactive (A1) astrocytes in various neurodegenerative diseases including Parkinson's disease (PD) and Alzheimer's disease (AD) (Liddelow, S. A. et al. Nature 541, 481-487, doi:10.1038/nature21029 (2017)). The abnormal misfolding and aggregation of α-synuclein and amyloid-β induce toxic effects in neurons in PD and AD, respectively. Therefore, development of agents that can inhibit the formation of M1-like microglia and reactive astrocytes can be developed as a universal neuroprotective drug for neurodegenerative disorders including PD and AD.

Embodiments of the invention are based, in part, on the discovery that α-synuclein and amyloid-β aggregates induce microglial activation and facilitate A1 astrocyte formation by secreting neurotoxic cytokines including TNFα, IL-1α, IL-1β, C1q and IL-6. Consequently, such inflammatory mediators released from activated microglia or reactive astrocytes causes neuronal damage and contribute to the progression of neurodegenerative diseases. Therefore, activated microglia and reactive astrocytes can be described as major upstream activities in neurodegenerative diseases. Inhibition of microglia activation and reactive astrocyte formation is a logical strategy to prevent, stop and/or reverse neurodegeneration processes. However, the lack of translational methods to specifically target microglia activation hampers this strategy.

The embodiments herein describe a unique strategy to target and block microglia activation and reactive astrocyte formation and the release of inflammatory and neurotoxic molecules from activated resident innate immune cells; thus prevent, stop, and/or ameliorate the progression of neurodegenerative diseases. In some embodiments, such methods can also be selective, for example, substantially not inhibiting normal functions of other cells in the CNS such as neurons so as to cause toxicity.

As detailed herein, RNA-sequencing analysis was performed and it was discovered that α-synuclein and amyloid-β aggregates-activated microglia significantly induce RIPK2 (receptor-interacting serine/threonine-protein kinase 2), an enzyme that in humans is encoded by the RIPK2 gene (Silke J et al., Nat Immunol. 16(7):689-97 (2015)) and NOD1 (nucleotide-binding oligomerization domain-containing protein 1) as well as NOD2. Surprisingly, it was found that depletion of RIPK2 and NOD2 in microglia significantly suppressed microglial activation and release of neurotoxic cytokines: thus inhibiting A1 astrocyte formation and protecting neurons.

Importantly, it was discovered that NOD2, RIPK2 and phosphorylated RIPK2 (p-RIPK2) levels are significantly increased in human postmortem brain tissues from patients with PD and AD compared to that of normal subjects. Moreover, increased p-RIPK2 signals are highly co-localized with microglia in the brain tissues from PD and AD patients as evident by immunohistochemistry. This suggests that RIPK2 activation play a pivotal role in the pathogenesis of neurodegenerative diseases including PD and AD and can be a clinically relevant therapeutic target.

In addition, when NOD2 and RIPK2 knock-out (KO) mice were induced PD by stereotaxic injection of α-synuclein preformed fibrils (α-synuclein PFFs), NOD2 and RIPK2 KO mice demonstrated significantly ameliorated LB/LN-like pathology, dopaminergic degeneration in mouse brain, and motor dysfunction, as well as reduced microglial activation and A1 astrocyte formation with protected neurons compared to that of α-synuclein PFFs-induced PD mice.

Similarly, NOD2 and RIPK2 KO mice induced AD by intracerebroventricular injection of amyloid-β aggregates demonstrated clearly improved memory functions and ameliorated cognitive deficits compared to normal amyloid-β-induced AD mice.

Furthermore, it was found that inhibition of RIPK2 activities by various orally active, small molecule-based RIPK2 inhibitors (1) inhibits α-synuclein PFFs-induced or amyloid-β aggregates-induced microglial activation, (2) blocks reactive astrocyte formation, and (3) finally secures neurons. Prior to the invention described herein, the role of RIPK2 and the effect of RIPK2 inhibitors in microglial activation and formation of reactive astrocytes were not known.

Lastly, it was confirmed that the oral administration of gefitinib, a known RIPK2 inhibitor, in α-synuclein PFFs-induced PD mice significantly rescues α-synuclein PFFs induced pathologies in mice while inhibiting microglial and astrocyte activation in vivo. Overall, these findings clearly provide evidence that RIPK2 is a viable therapeutic target for neurodegenerative disorders including PD and AD.

Accordingly, in certain embodiments, agents that inhibit microglial activation and/or the formation of reactive astrocytes by targeting RIPK2 and NOD2 will have profound therapeutic potential for PD and AD as disease-modifying therapies.

### Receptor Interacting Protein (RIP) Kinases

Receptor-interacting protein (RIP) kinases are a group of threonine/serine protein kinases with a relatively conserved kinase domain but distinct non-kinase regions. In humans, five different RIP kinase forms are known, designated RIP1, RIP2, RIP3, RIP4, and RIP5. A number of different domain structures, such as death domain and caspase activation and recruitment domain (CARD), were found in different RIP family members, and these domains have been considered as key features in determining the specific function of each RIP kinase. It is known that RIP kinases participate in different biological processes, including those in innate immunity, but their downstream substrates are largely unknown. Recent evidence has shown that the signaling pathway of necroptosis, a programmed form of necrosis, depends on the activation of RIP1 and RIP3 in response to death receptors induction. Direct cleavage of the RIPs by caspases prevents necroptotic cell death and it is associated with apoptotic cell death. It was recently shown that RIP1 and RIP3, in addition to their role in necroptosis, contribute to inflammation by activation of the NLRP3 inflammasome in dendritic cells (Kang, T. B. et al., Immunity; 38:27-40; 2013).

Receptor-interacting serine/threonine-protein kinase 2 (Accession number NP_003812; NCBI/Protein accession number NP_003812.1; gene accession number NM_003821) transduces signaling downstream of the intracellular peptidoglycan sensors NOD1 and NOD2 to promote a productive inflammatory response. However, excessive NOD2 signaling has been associated with numerous diseases, including inflammatory bowel disease (IBD), sarcoidosis, and inflammatory arthritis.

The nucleotide-binding oligomerization domain-containing proteins NOD1 and NOD2 are cytosolic Nod-like receptor (NLR) family proteins that function in the innate immune system to detect pathogenic bacteria (Philpott et al. Nat. Rev. Immunol., 14 (2014), pp. 9-23, 2014). NOD1 is activated upon binding to bacterial peptidoglycan fragments containing diaminopimelic acid (DAP), whereas NOD2 recognizes muramyl dipeptide (MDP) constituents (Chamaillard et al., Nat. Immunol., 4 (2003), pp. 702-707; Girardin et al., Science, 300 (2003), pp. 1584-1587; Girardin et al., J. Biol. Chem., 278 (2003), pp. 8869-8872; Inohara et al., J. Biol. Chem., 278 (2003), pp. 5509-5512). NOD activation induces pro-inflammatory signaling by receptor-interacting protein kinase 2 (RIPK2, also known as RIP2 or RICK), which plays an obligatory and specific role in activation of NOD-dependent, but not Toll-like receptor responses (Park et al., J. Immunol., 178 (2007), pp. 2380-2386).

Signaling by RIPK2 is dependent on an N-terminal kinase domain with dual Ser/Thr and Tyr kinase activities (Dorsch et al. Cell. Signal., 18 (2006), pp. 2223-2229; Tigno-Aranjuez et al., Genes Dev., 24 (2010), pp. 2666-2677), as well as a C-terminal caspase activation and recruitment domain (CARD) that mediates CARD-CARD domain assembly with activated NODs (Inohara et al., J. Biol. Chem., 274 (1999), pp. 14560-14567; Ogura et al., J. Biol. Chem., 276 (2001), pp. 4812-4818). Once engaged, RIPK2 is activated by autophosphorylation (Dorsch et al., 2006) and further targeted by XIAP (X-linked inhibitor of apoptosis) and other E3 ligases for non-degradative polyubiquitination (Bertrand et al., PLoS One, 6 (2011), p. e22356; Damgaard et al., Mol. Cell, 46 (2012), pp. 746-758; Tao et al., Curr. Biol., 19 (2009), pp. 1255-1263; Tigno-Aranjuez et al., Mol. Cell. Biol., 33 (2013), pp. 146-158; Yang et al., J. Biol. Chem., 282 (2007), pp. 36223-36229; Yang et al., Nat. Immunol., 14 (2013), pp. 927-936). The ubiquitin-conjugated protein subsequently activates the TAK1 and IKK kinases, leading to upregulation of both the mitogen-activated protein kinase and nuclear factor κB (NF-κB) signaling pathways (Kim et al., J. Biol. Chem., 283 (2008), pp. 137-144; Park et al., J. Immunol., 178 (2007), pp. 2380-2386). In addition, RIPK2 induces an antibacterial autophagic response by signaling between NODs and the autophagy factor ATG16L1 (Cooney et al., Nat. Med., 16 (2010), pp. 90-97; Homer et al., J. Biol. Chem., 287 (2012), pp. 25565-25576).

### Ripk2 Inhibitors

Inhibition of RIPK2 activity is typically mediated by at least one or more of: reducing, inhibiting or preventing the expression of RIPK2, neutralizing the functionality of RIPK2, and inducing RIPK2 degradation. According to certain embodiments, inhibiting RIPK2 activity is mediated by reducing, inhibiting or preventing the expression of RIPK2. Inhibiting RIPK2 activity can be mediated directly by interacting with the RIPK2 protein, gene or mRNA or indirectly by interacting with a protein, gene or mRNA associated with RIP-mediated activity or expression.

Different categories of RIPK2 inhibitors are suitable for the compositions and methods herein, which include but are not limited to small molecules, antibodies, nucleic acid molecules (DNAs, RNAs such as shRNA, siRNA, antisense molecules, etc.), etc., which can inhibit the expression, processing, post-translational modification, or activity of RIPK2 or a molecule in a biological pathway involving RIPK2. In some embodiments, a RIPK2 inhibitor can inhibit (e.g., specifically inhibit) the expression, processing, post-translational modification, or activity of RIPK2. In other embodiments, a RIPK2 inhibitor can inhibit (e.g., specifically inhibit) the expression, processing, post-translational modification, or activity of unspliced RIPK2 gene.

In some embodiments, RIPK2 inhibitors of the invention can be, for example, intracellular binding molecules that act to specifically or directly inhibit the expression, processing, post-translational modification, or activity, e.g., of RIPK2 or a molecule in a biological pathway involving RIPK2. As used herein, the term “intracellular binding molecule” is intended to include molecules that act intracellularly to inhibit the processing expression or activity of a protein by binding to the protein or to a nucleic acid (e.g., an mRNA molecule) that encodes the protein. Examples of intracellular binding molecules, described in further detail below, include antisense nucleic acids, intracellular antibodies, peptidic compounds that inhibit the interaction of RIPK2 or a molecule in a biological pathway involving RIPK2 and chemical agents that specifically or directly inhibit RIPK2 activity or the activity of a molecule in a biological pathway involving RIPK2.

In some embodiments, RIPK2 inhibitors can be enzymatic nucleic acids. Expression of a given gene can be inhibited by an enzymatic nucleic acid. As used herein, an “enzymatic nucleic acid” refers to a nucleic acid comprising a substrate binding region that has complementarity to a contiguous nucleic acid sequence of a gene, and which is able to specifically cleave the gene. The enzymatic nucleic acid substrate binding region can be, for example, 50-100% complementary, 75-100% complementary, 90-100% complementary, or 95-100% complementary to a contiguous nucleic acid sequence in a gene. The enzymatic nucleic acids can also comprise modifications at the base, sugar, and/or phosphate groups. An exemplary enzymatic nucleic acid for use in the present methods is a ribozyme. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, and RNAzyme.

Small Molecules:

In certain embodiments, the RIPK2 inhibitor can comprise one or more small molecules that inhibit (e.g., selectively inhibit) RIPK2. Suitable small molecule RIPK2 inhibitors include any of those known in the art. For example, in certain embodiments, the small molecule can be Gefitinib (IRESSA™, AstraZeneca), SB203580 (Gretchen M. Argast et al., Mol. Cell. Biochem. Vol. 268, 129-140 (2005)), OD36, OD38 (J. T. Tigno-Aranjuez et al., J. Biol Chem. Vol. 289 No. 43, 29651-29664 (2014)), ponatinib, sorafenib, regorafenib or GSK583 (Pamela A Haile et al., J. Med. Chem. Vol 59 N. 10, 4867-4880 (2016)), and pharmaceutically acceptable salts thereof. In some embodiments, the RIPK2 inhibitor has an IC₅₀ value similar to (within 5-fold) or better than the IC₅₀ value observed for Gefitinib in an in vitro RIPK2 kinase assay.

Non-limiting useful small molecule RIPK2 inhibitors also include any of those described in the following U.S. or PCT application publications: US20160024114A1; WO2011106168A1; US2013/0251702A1; US20180118733A1; WO2016042087A1; WO2018052773A1; WO2018052772A1; WO2011112588A2; WO2011120025A1; WO2011120026A1; WO2011123609A1; WO2011140442A1; WO2012021580A1; WO2012122011A2; WO2013025958A1; WO2014043437A1; WO2014043446A1; WO2014128622A1; WO2016172134A2; WO2017046036A1; WO2017182418A1; WO2012003544A1; the content of each of which is herein incorporated by reference in its entirety.

Non-limiting suitable small molecule RIPK2 inhibitors can also include any of those described in the following: Cruz J. V., et al., “Identification of Novel protein kinase receptor type 2 inhibitors using pharmacophore and structure-based virtual screening,” Molecules 23, 453, pages 1-25 (2018); Sala M., et al., “Identification and characterization of novel receptor-interacting serine/threonine-protein kinase 2 inhibitors using structural similarity analysis, The Journal of Pharmacology and Experimental Therapeutics, 365:354-367 (2018); He X, et al., “Identification of potent and selective RIPK2 inhibitors for the treatment of inflammatory diseases,” ACS Med Chem Lett 8:1048-1053(2017); the content of each of which is herein incorporated by reference in its entirety.

In some embodiments, the RIPK2 inhibitor can also be a CSLP molecule:

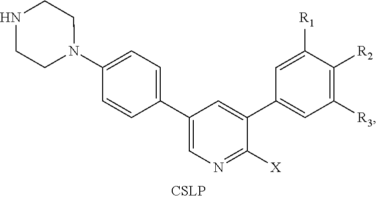

wherein:

X is methyl or NH₂,

R₁ is hydrogen, F, or methoxy,

R₂ is hydrogen, hydroxyl, or methoxy, and

R₃ is —NHSO₂(n-propyl), or a pharmaceutically acceptable salt thereof. Examples of CSLP molecules as RIPK2 inhibitors have been described, see, e.g., Hrdinka M. et al., The EMBO Journal, e99372, pages 1-16 (2018), the content of which is herein incorporated by reference in its entirety. In some embodiments, the RIPK2 inhibitor can also be a CSLP molecule or a pharmaceutically acceptable salt thereof, wherein X is NH₂, R₁ is methoxy, R₂ is methoxy, and R₃ is —NHSO₂(n-propyl). In some embodiments, the RIPK2 inhibitor can also be a CSLP molecule or a pharmaceutically acceptable salt thereof, wherein X is NH₂, R₁ is F, R₂ is methoxy, and R₃ is —NHSO₂(n-propyl).

In any of the embodiments described herein, the RIPK2 inhibitor can also be gefitinib, sorafenib, regorafenib, ponatinib, SB203580, OD36 (6-Chloro-10,11,14,17-tetrahydro-13H-1,16-etheno-4,8-metheno-1H-pyrazolo[3,4-g][1,14,4,6]dioxadiazacyclohexadecine), OD38 ([4,5,8,9-Tetrahydro-7H-2,17-etheno-10,14-metheno-1H-imidazo[1,5-g][1,4,6,7,12,14]oxapentaazacyclohexadecine]), WEHI-435(N-(2-(4-amino-3-(p-tolyl)-1H-pyrazolo[3,4-d]pyrimidin-1-yl)-2-methylpropyl)isonicotinamide), or GSK583 (6-(tert-butylsulfonyl)-N-(5-fluoro-1H-indazol-3-yl)quinolin-4-amine) or a pharmaceutically acceptable salt thereof. In some specific embodiments, the RIPK2 inhibitor can be gefitinib or GSK583 or a pharmaceutically acceptable salt thereof.

In certain embodiments, the small molecule RIPK2 inhibitors can inhibit one or more pathways that the RIP kinases are involved with. For example, RIPK2 kinase is integral to NOD2 activation, including the initiation of downstream NF-κB, MAPK, and autophagy pathways (J. T. Tigno-Aranjuez et al., J. Biol Chem. Vol. 289 No. 43, 29651-29664 (2014); Kobayashi K., et al., Nature 416, 194-199 (2002); Park J. H., et al., J. Immunol. 178, 2380-2386 (2007); Homer C. R., et al., J. Biol. Chem. 287, 25565-25576 18-20 (2012)). Useful small molecules RIPK2 inhibitors can be identified by one or more assays, as exemplified below.

Antisense Oligonucleotides:

In some embodiments, the RIPK2 inhibitor is an antisense nucleic acid molecule that is complementary to a gene encoding RIPK2 or a molecule in a pathway involving RIP kinase (e.g., a molecule with which RIPK2 interacts), or to a portion of such a gene, or a recombinant expression vector encoding an antisense nucleic acid molecule. Some examples of RIPK2 antisense are described in U.S. Pat. No. 6,426,221, the content of which is herein incorporated by reference in its entirety. The use of antisense nucleic acids to downregulate the expression of a particular protein in a cell is well known in the art (see e.g., Weintraub, H., et al. 1986. Reviews—Trends in Genetics, Vol. 1(1); Askari, F. K., et al. 1996. N. Eng. Med. 334, 316-318; Bennett, M. R., et al. 1995. Circulation 92, 1981-1993; Mercola, D., et al. 1995. Cancer Gene Mer. 2, 47-59; Rossi, J. J., 1995. Br. Med. Bull. 51, 217-225; Wagner. R. W., 1994. Nature 372, 333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. In one embodiment, an antisense nucleic acid is designed so as to be complementary to a region preceding or spanning the initiation codon on the coding strand or in the 3′ untranslated region of an mRNA. Given the known nucleotide sequence for the coding strand of the RIP kinase gene and thus the known sequence of the RIP kinase mRNA, antisense nucleic acids of the invention can be designed according to the rules of Watson and Crick base pairing. For example, the antisense oligonucleotide can be complementary to the region surrounding the translation start site of an RIP kinase, an antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. To inhibit expression in cells, one or more antisense oligonucleotides can be used.

Alternatively, an anti-sense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). The antisense expression vector can be in the form of, for example, a recombinant plasmid, phagemid or attenuated virus. The antisense expression vector can be introduced into cells using a standard transfection technique.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet other embodiments, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gautier, C., et al. 1987. Nucleic Acids. Res. 15, 6625-6641). The antisense nucleic acid molecule can also comprise a 2′-O-methylribonucleotide (Inoue, H., et al. 1987. Nucleic Acids Res. 15, 6131-6148) or a chimeric RNA-DNA analogue (Inoue, H., et al. 1987. FEBS Lett. 215, 327-330).

In still other embodiments, an antisense nucleic acid molecule of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff, J., et al. 1988. Nature 334, 585-591)) can be used to catalytically cleave mRNA transcripts to thereby inhibit translation mRNAs. Alternatively, gene expression can be inhibited by targeting nucleotide sequences complementary to the regulatory region of a gene (e.g., RIP kinase promoter and/or enhancer) to form triple helical structures that prevent transcription of a gene in target cells. See generally, Helene, C., 1991. Anticancer Drug Des. 6(6), 569-84; Helene, C., et al. 1992. Ann. N.Y. Acad. Sci. 660, 27-36; and Maher, L. J., 1992. Bioassays 14(12), 807-15.

In other embodiments, a compound that promotes RNAi can be used to inhibit expression of any one or more RIP kinases or a molecule in a biological pathway involving RIP kinases. The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes. RNA interference (RNAi is a post-transcriptional, targeted gene-silencing technique that uses double-stranded RNA (dsRNA) to degrade messenger RNA (mRNA) containing the same sequence as the dsRNA (Sharp, P. A., et al. 2000. Science 287, 5462:2431-3; Zamore, P. D., et al. 2000. Cell 101, 25-33. Tuschl, T., et al. 1999. Genes Dev. 13, 3191-3197; Cottrell T. R., et al., 2003. Trends Microbiol. 11, 37-43; Bushman F., 2003. Mol. Therapy 7, 9-10; McManus M. T., et al. 2002. Nat Rev Genet 3, 737-47). The process occurs when an endogenous ribonuclease cleaves the longer dsRNA into shorter, e.g., 21-23-nucleotide-long RNAs, termed small interfering RNAs or siRNAs. As used herein, the term “small interfering RNA” (“siRNA”) (also referred to in the art as “short interfering RNAs”) refers to an RNA agent, such as a double-stranded agent, of about 10-50 nucleotides in length (the term “nucleotides” including nucleotide analogs), e.g., between about 15-25 nucleotides in length, or about 17, 18, 19, 20, 21, 22, 23, 24, or 25 nucleotides in length, the strands optionally having overhanging ends comprising, e.g., 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof). The smaller RNA segments then mediate the degradation of the target mRNA. Kits for synthesis of RNAi are commercially available from, e.g. New England Biolabsor Ambion. In some embodiments, one or more of the chemistries described above for use in antisense RNA can be employed in molecules that mediate RNAi.

Alternatively, compound that promotes RNAi can be expressed in a cell, e.g., a cell in a subject, to inhibit expression of RIP kinases or a molecule in a biological pathway involving RIP kinases. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway. The requisite elements of a shRNA molecule include a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. In certain embodiments, shRNAs of the invention include the sequences of a desired siRNA molecule described supra. In such embodiments, shRNA precursors include in the duplex stem the 21-23 or so nucleotide sequences of the siRNA, desired to be produced in vivo.

Efficient delivery to cells in vivo requires specific targeting and substantial protection from the extracellular environment, particularly serum proteins. One method of achieving specific targeting is to conjugate a targeting moiety to the iRNA agent. The targeting moiety helps in targeting the iRNA agent to the required target site. One way a targeting moiety can improve delivery is by receptor mediated endocytotic activity. This mechanism of uptake involves the movement of iRNA agent bound to membrane receptors into the interior of an area that is enveloped by the membrane via invagination of the membrane structure or by fusion of the delivery system with the cell membrane. This process is initiated via activation of a cell-surface or membrane receptor following binding of a specific ligand to the receptor. Many receptor-mediated endocytotic systems are known and have been studied, including those that recognize sugars such as galactose, mannose, mannose-6-phosphate, peptides and proteins such as transferrin, asialoglycoprotein, vitamin B12, insulin and epidermal growth factor (EGF). The Asialoglycoprotein receptor (ASGP-R) is a high capacity receptor, which is highly abundant on hepatocytes. The ASGP-R shows a 50-fold higher affinity for N-Acetyl-D-Galactosylamine (GalNAc) than D-Gal. Previous work has shown that multivalency is required to achieve nM affinity, while spacing among sugars is also important.

The mannose receptor, with its high affinity to D-mannose represents another important carbohydrate-based ligand-receptor pair. The mannose receptor is highly expressed on specific cell types such as macrophages and possibly dendritic cells Mannose conjugates as well as mannosylated drug carriers have been successfully used to target drug molecules to those cells. For examples, see Biessen et al. (1996) J. Biol. Chem. 271, 28024-28030; Kinzel et al. (2003) J. Peptide Sci. 9, 375-385; Barratt et al. (1986) Biochim. Biophys. Acta 862, 153-64; Diebold et al. (2002) Somat. Cell Mol. Genetics 27, 65-74.

Lipophilic moieties, such as cholesterol or fatty acids, when attached to highly hydrophilic molecules such as nucleic acids can substantially enhance plasma protein binding and consequently circulation half-life. In addition, binding to certain plasma proteins, such as lipoproteins, has been shown to increase uptake in specific tissues expressing the corresponding lipoprotein receptors (e.g., LDL-receptor HDL-receptor or the scavenger receptor SR-B1). For examples, see Bijsterbosch, M. K., Rump, E. T. et al. (2000) Nucleic Acids Res. 28, 2717-25; Wolfrum, C., Shi, S. et al. (2007) 25, 1149-57. Lipophilic conjugates can also be used in combination with the targeting ligands in order to improve the intracellular trafficking of the targeted delivery approach.

PULMOZYME™ is provided as a liquid protein formulation ready for use in nebulizer systems. In addition to nebulizer systems, pulmonary administration of drugs and other pharmaceuticals can be accomplished by provision of an inhalable solution formulated for inhalation by means of suitable liquid-based inhalers known as metered dosage inhalers or a dry powder formulation for inhalation by means of suitable inhalers known as dry powder inhalers (DPIs).

Intracellular Antibodies:

Another type of inhibitory compound that can be used to inhibit the expression and/or activity of RIP kinase or a molecule in a biological pathway involving RIP kinase is an intracellular antibody specific for said protein. The use of intracellular antibodies to inhibit protein function in a cell is known in the art (see e.g., Carlson, J. R., 1988. Mol. Cell. Biol. 8, 2638-2646; Biocca, S., et al. 1990. EMBO. J. 9, 101-108; Werge, T. M., et al. 1990. FEBS Letters 274, 193-198; Carlson, J. R., 1993. Proc. Natl. Acad. Sci. USA 90, 7427-7428; Marasco, W. A., et al., 1993. Proc. Natl. Acad. Sci. USA 90, 7889-7893; Biocca, S., et al. 1994. BioTechnology 12, 396-399; Chen, S. Y., et al. 1994. Human Gene Therapy 5, 595-601; Duan, L., et al. 1994. Proc. Natl. Acad. Sci. USA 91, 5075-5079; Chen, S. Y., et al. 1994. Proc. Natl. Acad. Sci. USA 91, 5932-5936; Beerli, R. R., et al. 1994. J. Biol. Chem. 269, 23931-23936; Beerli, R. R., et al. 1994. Biochem. Biophys. Res. Commun. 204, 666-672; Mhashilkar, A. M., et al. 1995. EMBO J 14, 1542-1551; Richardson, J. H., et al. 1995. Proc. Natl. Acad. Sci. USA 92, 3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of RIP kinase activity according to the methods of the invention an intracellular antibody that specifically binds the protein is expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N- or C-terminus of the light and heavy chain genes (see e.g., Biocca. S., et al. 1990. EMBO J. 9, 101-108; Mhashilkar, A. M., et al. 1995. EMBO. J. 14, 1542-1551). A nuclear localization signal that can be used for nuclear targeting of the intracellular antibody chains is the nuclear localization signal of SV40 Large T antigen (see Biocca, S., et al. 1990. EMBO J. 9, 101-108; Mhashilkar, A. M., et al. 1995. EMBO J. 14, 1542-1551).

Gene Editing Agents:

In certain embodiments, the inhibitor is a gene editing agent. The gene editing agent can inactivate or remove the entire gene or portions thereof to inhibit or prevent transcription and translation. Any suitable nuclease system can be used including, for example, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J. Virol. 88(17):8920-8936, incorporated herein by reference in its entirety.

In certain embodiments, the gene editing agent is a Clustered Regularly Interspaced Short Palindromic Repeat (CRISPR)-associated endonuclease/Cas (CRISPR/Cas). The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the protein. The CRISPR/Cas-like protein can also be truncated or modified to optimize the activity of the effector domain of the protein. In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

In embodiments, the CRISPR/Cas system can be a type I, a type II, or a type III system. Non-limiting examples of suitable CRISPR/Cas proteins include Cas3, Cas4, Cas5, Cas5e (or CasD), Cash, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the RNA-guided endonuclease is derived from a type II CRISPR/Cas system. In other embodiments, the RNA-guided endonuclease is derived from a Cas9 protein.

In certain embodiments, the system is an Argonaute nuclease system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways (Swarts, D.C. et al. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss) RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). Guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

Argonaute proteins typically have a molecular weight of ˜100 kDa and are characterized by a Piwi-Argonaute-Zwille (PAZ) domain and a PIWI domain. Crystallographic studies of archaeal and bacterial Argonaute proteins revealed that the PAZ domain, which is also common to Dicer enzymes, forms a specific binding pocket for the 3′-protruding end of the small RNA with which it associates (Jinek and Doudna, (2009) Nature 457, 405-412)). The structure of the PIWI domain resembles that of bacterial RNAse H, which has been shown to cleave the RNA strand of an RNA-DNA hybrid (Jinek and Doudna, (2009) Nature 457, 405-412)). More recently, it was discovered that the catalytic activity of miRNA effector complexes, also referred to as Slicer activity, resides in the Argonaute protein itself.

Members of the human Ago subfamily, which consists of AGO1, AGO2, AGO3 and AGO4, are ubiquitously expressed and associate with miRNAs and siRNAs. Ago proteins are conserved throughout species, and many organisms express multiple family members, ranging from one in Schizosaccharomyces pombe, five in Drosophila, eight in humans, ten in Arabidopsis to twenty-seven in C. elegans (Tolia and Joshua-Tor, (2007) Nat. Chem. Biol. 3, 36-43). Argonaute proteins are also present in some species of budding yeast, including Saccharomyces castellii. It was found that S. castellii expresses siRNAs that are produced by a Dicer protein that differs from the canonical Dicer proteins found in animals, plants and other fungi (Drinnenberg et al., (2009) Science 326, 544-550).

Structural studies have been extended to Thermus thermophilus Argonaute in complex with a guide strand only or a guide DNA strand and a target RNA duplex. This analysis revealed that the structure of the complex is divided into two lobes. One lobe contains the PAZ domain connected to the N-terminal domain through a linker region, L1. The second lobe consists of the middle (MID) domain (located between the PAZ and the PIWI domains) and the PIWI domain. The 5′ phosphate of the small RNA, to which Argonaute binds, is positioned in a specific binding pocket in the MID domain (Jinek and Doudna, (2009) Nature 457, 405-412). The contacts between the Argonaute protein and the guide DNA or RNA molecule are dominated by interactions with the sugar-phosphate backbone of the small RNA or DNA; thus, the bases of the RNA or DNA guide strand are free for base pairing with the complementary target RNA. The structure indicates that the target mRNA base pairs with the guide DNA strand, but does not touch the protein (Wang et al., (2008a) Nature 456, 921-926; Wang, Y. et al., (2009) Nat. Struct. Mol. Biol. 16, 1259-1266; Wang et al., (2008b) Nature 456, 209-213).

The useful features of Argonaute endonucleases, e.g. Natronobacterium gregoryi Argonaute (NgAgo) for genome editing include the following: (i) NgAgo has a low tolerance to guide-target mismatch; (ii) 5′ phosphorylated short ssDNAs are rare in mammalian cells, which minimizes the possibility of cellular oligonucleotides misguiding NgAgo; and (iii) NgAgo follows a “one-guide-faithful” rule, that is, a guide can only be loaded when NgAgo protein is in the process of expression, and, once loaded, NgAgo cannot swap its gDNA with other free ssDNA at 37° C.

Accordingly, in certain embodiments, Argonaute endonucleases comprise those which associate with single stranded RNA (ssRNA) or single stranded DNA (ssDNA). In certain embodiments, the Argonaute is derived from Natronobacterium gregoryi. In other embodiments, the Natronobacterium gregoryi Argonaute (NgAgo) is a wild type NgAgo, a modified NgAgo, or a fragment of a wild type or modified NgAgo. The NgAgo can be modified to increase nucleic acid binding affinity and/or specificity, alter an enzymatic activity, and/or change another property of the protein. For example, nuclease (e.g., DNase) domains of the NgAgo can be modified, deleted, or inactivated.

Other inhibitory agents that can be used to specifically inhibit the activity of an RIP kinase or a molecule in a biological pathway involving RIP kinase are chemical compounds that directly inhibit expression, processing, post-translational modification, and/or activity of, e.g., an RIP kinase-2. Such compounds can be identified using screening assays that select for such compounds, as described in detail as well as using other art recognized techniques.

In exemplary embodiments, one or more of the above-described inhibitory compounds is formulated according to standard pharmaceutical protocols to produce a pharmaceutical composition for therapeutic use. A pharmaceutical composition of the invention is formulated to be compatible with its intended route of administration.

### Screening Assays

In certain aspects, the invention features methods for identifying compounds useful in inhibiting the RIP kinases. In certain embodiments, the inhibitor is an inhibitor of RIPK2. Examples of screening assays include, without limitation gene expression assays, transcriptional assays, kinase assays, immune assays, and the like.

Small molecules for screening as inhibitors of RIP kinases, can be obtained from commercially available libraries, for example, NANOCYCLIX® (Oncodesign). Screening of compound libraries can be performed using in vitro radiometric kinase assays utilizing recombinantly purified RIPK2 expressed in cells, such as insect cells, as kinase and RBER-CHKtide as a substrate. Various concentrations of inhibitor can be tested ranging from 3×10⁻⁶ m to 9×10⁻¹¹ m using about 50 ng recombinant RIPK2 and 2 μg of recombinant RBER-CHKtide substrate per 50 μl reaction. Compounds which show in vitro IC₅₀ values of <100 nm are then tested in a cellular assay where RIPK2 activity (tyrosine autophosphorylation) is induced by co-expression of NOD2 with RIPK2 and inhibition of kinase activity assessed by loss of tyrosine autophosphorylation upon treatment with RIPK2 inhibitor. The compounds which maintain inhibition of RIPK2 tyrosine phosphorylation in the cellular assay at the lower, e.g. about 250 nm dose are then used for further in vitro and in vivo assays. Kinase specificity can be tested by pre-incubation of recombinant kinase with various doses of inhibitor before conducting an in vitro kinase assay using a known substrate. After 30 min, the reaction is stopped and phosphate incorporation is measured.

Accordingly, in exemplary aspects the invention features methods of identifying compounds useful in inhibiting the phosphorylation activity of RIP kinases. This can include, inhibition of transcription, translation, gene expression, activity and the like of RIP kinases. In exemplary aspects, the methods comprise: providing an indicator composition comprising a purified recombinant RIP kinase and a substrate; contacting the indicator composition with each member of a library of test compounds; and selecting from the library of test compounds a compound of interest that decreases the kinase activity.

In other embodiments, a screening assay measures the effect of an inhibitor on: (1) NOD1 and NOD2-dependent activation of NF-kB, which plays a critical role in inflammation, (2) amyloid-beta and alpha-synuclein aggregates-induced microglial activation and blocking of A1 astrocyte formation and (3) maintenance of neurons.

As used herein, the term “test compound” refers to a compound that has not previously been identified as, or recognized to be, a modulator of the activity being tested. The term “library of test compounds” refers to a panel comprising a multiplicity of test compounds. As used herein, the term “indicator composition” refers to a composition that includes a protein of interest (e.g., RIPK2 or a molecule in a biological pathway involving RIPK2, e.g., NOD1, NOD2), for example, a cell that naturally expresses the protein, a cell that has been engineered to express the protein by introducing one or more of expression vectors encoding the protein(s) into the cell, or a cell free composition that contains the protein(s) (e.g., purified naturally-occurring protein or recombinantly-engineered protein(s)). The term “cell” includes prokaryotic and eukaryotic cells. In some embodiments, a cell of the invention is a bacterial cell. In other embodiments, a cell of the invention is a fungal cell, such as a yeast cell. In other embodiments, a cell of the invention is a vertebrate cell, e.g., an avian or mammalian cell. In other embodiments, a cell of the invention is a murine or human cell. As used herein, the term “engineered” (as in an engineered cell) refers to a cell into which a nucleic acid molecule e.g., encoding an RIP kinase (e.g., a spliced and/or unspliced form) has been introduced.

In some embodiments, the present invention also provides a method of identifying a therapeutic agent for a neurodegenerative disease or disorder (e.g., those associated with upregulated NOD2, phosphorylated RIPK2, and/or RIPK2 in one or more regions of the central nervous system (CNS)). In some embodiments, the method comprises contacting a CNS resident innate immune cell with an agent that induces the activation of the immune cell (e.g., an abnormally aggregated protein) in the presence of a candidate therapeutic agent; measuring activation of the CNS resident innate immune cell in the presence of the candidate therapeutic agent; and identifying a therapeutic agent that inhibits activation of the CNS resident innate immune cell compared to a control. In some embodiments, the candidate therapeutic agent is a RIPK2 inhibitor, e.g., identified by a screening assay herein. In some embodiments, contacting the CNS resident innate immune cell with the agent induces upregulation of NOD2, phosphorylated RIPK2, and/or RIPK2. In some embodiments, the CNS resident innate immune cell is microglia and/or astrocyte. In some embodiments, the agent that induces the activation of the CNS resident innate immune cell is an abnormally aggregated protein such as α-synuclein, amyloid-β, and/or tau. In some embodiments, the measuring comprises measuring expression level of NOD2, phosphorylated RIPK2, and/or RIPK2. In some embodiments, the measuring comprises measuring expression level of factors iNOS, Cxcl1, and/or IL-1β. In some embodiments, the measuring comprises measuring chemotaxis of the CNS immune cell. In any of such embodiments, the method can comprise identifying a therapeutic agent that inhibits RIPK2 activity and/or expression, e.g., selectively inhibits RIPK2 activity and/or expression over other RIP kinases; inhibits NOD2-dependent activation of NF-kB; and/or inhibits amyloid-β aggregates-induced microglial activation, alpha-synuclein aggregates-induced microglial activation and/or A1 astrocyte formation. In any of such embodiments, the neurodegenerative disease or disorder can be Alzheimer's disease, amyotropic lateral sclerosis (ALS/Lou Gehrig's Disease), Parkinson's disease, diabetic neuropathy, polyglutamine (polyQ) diseases, stroke, Fahr disease, multiple sclerosis, Menke's disease, Wilson's disease, cerebral ischemia, a prion disorder, dementia, corticobasal degeneration, progressive supranuclear palsy, multiple system atrophy, hereditary spastic paraparesis, spinocerebellar atrophies, brain injury, and/or spinal cord injury. In some specific embodiments, the neurodegenerative disease or disorder can be Alzheimer's disease or Parkinson's disease. In some embodiments, the present invention is also directed to the therapeutic agent identified with any of the screening methods herein.

### Pharmaceutical Compositions

Additional aspects provide pharmaceutical compositions comprising a RIPK2 inhibitor as an active agent and a pharmaceutically acceptable carrier, excipient or diluent. Any of the RIPK2 inhibitors described herein are suitable. In some embodiments, the RIPK2 inhibitor is the only active ingredient in the pharmaceutical composition. In some embodiments, the RIPK2 and one or more additional active ingredients (e.g., described herein) can be included in the pharmaceutical composition.

The RIPK2 inhibitors can be formulated depending on the route of administration. In certain embodiments, the RIPK2 inhibitor is administered via a route of administration comprising: intravenously, subcutaneously, intra-arterially, intraperitoneally, ophthalmically, intramuscularly, buccally, rectally, vaginally, intraorbitally, intracerebrally, intradermally, intracranially, intraspinally, intraventricularly, intrathecally, intracisternally, intracapsularly, intrapulmonary, intranasally, transmucosally, transdermally, inhalation, or any combination thereof.

In certain embodiments, the RIPK2 inhibitor is administered orally or parenterally.

In certain embodiments of the present invention, the RIPK2 inhibitor(s) therapeutic agent(s) is administered in a dosage form that permits systemic uptake, such that the therapeutic agent(s) can cross the blood-brain barrier so as to exert effects on neuronal cells. For example, pharmaceutical formulations of the therapeutic agent(s) suitable for parenteral/injectable used generally include sterile aqueous solutions (where water soluble), or dispersions and sterile powders for the extemporaneous preparation of sterile injectable solutions or dispersion. In all cases, the form must be sterile and must be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, polyethylene glycol, and the like), suitable mixtures thereof, or vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate or gelatin.

Sterile injectable solutions are prepared by incorporating the therapeutic agent(s) in the required amount in the appropriate solvent with various other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation include vacuum drying and the freeze-drying technique, which yield a powder of the active ingredient plus any additional desired ingredient from previously sterile-filtered solution thereof. Pharmaceutical compositions according to the invention are typically liquid formulations suitable for injection or infusion. For example, saline solutions and aqueous dextrose and glycerol solutions can be employed as liquid carriers, particularly for injectable solutions.

Solutions or suspensions used for intravenous administration typically include a carrier such as physiological saline, bacteriostatic water, Cremophor (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride can be included in the composition. Prolonged absorption of the composition can be accomplished by adding an agent which delays absorption, e.g., aluminum monostearate and gelatin. Where necessary, the composition can also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Oral compositions include an inert diluent or edible carrier. The composition can be enclosed in gelatin or compressed into tablets. For the purpose of oral administration, the active agent can be incorporated with excipients and placed in tablets, troches, or capsules. Pharmaceutically compatible binding agents or adjuvant materials can be included in the composition. Tablets, troches, and capsules can optionally contain a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; or a sweetening agent or a flavoring agent.

The composition can also be administered by a transmucosal or transdermal route. Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. The composition can be formulated as a suppository, with traditional binders and carriers such as triglycerides.

Solutions or suspensions used for intradermal or subcutaneous application typically include at least one of the following components: a sterile diluent such as water, saline solution, fixed oils, polyethylene glycol, glycerin, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations can be enclosed in ampoules, disposable syringes, or multiple dose vials.

In certain embodiments, polypeptide active agents are prepared with carriers to protect the polypeptide against rapid elimination from the body. Biodegradable polymers (e.g., ethylene vinyl acetate, polyanhydrides, polyglycolic acid, collagen, polyorthoesters, and polylactic acid) are often used. Methods for the preparation of such formulations are known by those skilled in the art. Liposomal suspensions can be used as pharmaceutically acceptable carriers too. The liposomes can be prepared according to established methods known in the art (for example, U.S. Pat. No. 4,522,811).

The administered dose of the RIPK2 inhibitor in the method of the present invention can be determined while taking into consideration various conditions of a subject that requires treatment, for example, the severity of symptoms, general health conditions of the subject, age, weight, sex of the subject, diet, the timing and frequency of administration, a medicine used in combination, responsiveness to treatment, and compliance with treatment.

### Methods of Treatment

In various embodiments, the present invention also provides a method of preventing or treating a neurodegenerative disease or disorder such as Parkinson's disease or Alzheimer's disease, the method comprises administering to a subject (e.g., human) in need thereof, a therapeutically effective amount of a Receptor-Interacting Protein (RIP) kinase 2 (RIPK2) inhibitor or a pharmaceutical composition comprising a RIPK2 inhibitor. Any of the RIPK2 inhibitors and pharmaceutical compositions comprising the RIPK2 inhibitor as described herein can be used. For example, useful RIPK2 inhibitors include those that can inhibit the activity of RIPK2 and/or its expression. In some embodiments, the RIPK2 inhibitors can be selective inhibitors over other RIP kinases such as RIPK1 and/or RIPK3, for example, with a selectivity of about 2-fold, about 3-fold, about 4-fold, about 5-fold, about 10-fold, or higher. In some embodiments, the RIPK2 inhibitor has substantially no activity against other RIP kinases. However, in some embodiments, the RIPK2 inhibitor can also be a dual or multi RIP kinases inhibitor, or a pan-RIP kinase inhibitor.

In some embodiments, the neurodegenerative disease or disorder is associated with upregulated NOD2, phosphorylated RIPK2, and/or RIPK2 in one or more regions of the central nervous system (CNS). Various diseases or disorders associated with upregulated NOD2, phosphorylated RIPK2, and/or RIPK2 in the CNS can be treated with the methods herein. Non-limiting examples include Alzheimer's disease, amyotropic lateral sclerosis (ALS/Lou Gehrig's Disease), Parkinson's disease, diabetic neuropathy, polyglutamine (polyQ) diseases, stroke, Fahr disease, Menke's disease, Wilson's disease, cerebral ischemia, a prion disorder, dementia, corticobasal degeneration, progressive supranuclear palsy, multiple system atrophy, hereditary spastic paraparesis, spinocerebellar atrophies, brain injury, or spinal cord injury.

In some embodiments, the neurodegenerative disease or disorder is associated with activation of CNS resident innate immune cells. In some embodiments, the neurodegenerative disease or disorder is associated with activation of CNS resident innate immune cells, e.g., mediated by one or more abnormal proteins, such as an abnormal aggregated protein. In some embodiments, the CNS resident innate immune cells are microglia and/or astrocytes. In some embodiments, the abnormal protein comprises α-synuclein, amyloid-β, and/or tau. In some embodiments, the neurodegenerative disease or disorder is Parkinson's disease or Alzheimer's disease. In such embodiments, the RIPK2 inhibitor is typically administered in an amount effective to inhibit the activation of the CNS resident innate immune cells. In some embodiments, the RIPK2 inhibitor can be administered in an amount effective to reduce the level of one or more inflammatory or neurotoxic mediators (such as TNFα, IL-1α, IL-1β, C1q, and/or IL-6) secreted from the activated resident innate immune cells that induce neuro-inflammation and neuronal damage.

Certain specific embodiments are directed to a method of treating or preventing Parkinson's disease comprising administering to a subject (e.g., human) in need thereof a therapeutically effective amount of a RIPK2 inhibitor or a pharmaceutical composition comprising a RIPK2 inhibitor. Any of the RIPK2 inhibitors and pharmaceutical compositions comprising the RIPK2 inhibitor as described herein can be used. For example, useful RIPK2 inhibitors include those that can inhibit the activity of RIPK2 and/or its expression. In some embodiments, the RIPK2 inhibitors can be selective inhibitors over other RIP kinases such as RIPK1 and/or RIPK3, for example, with a selectivity of about 2-fold, about 4-fold, about 10-fold, or higher. In some embodiments, the RIPK2 inhibitor has substantially no activity against other RIP kinases. However, in some embodiments, the RIPK2 inhibitor can also be a dual or multi RIP kinases inhibitor, or a pan-RIP kinase inhibitor. In some embodiments, the RIPK2 inhibitor is a small molecule RIPK2 inhibitor described herein.

Certain embodiments are also directed to a method of treating or preventing Alzheimer's disease comprising administering to a subject (e.g., human) in need thereof a therapeutically effective amount of a RIPK2 inhibitor or a pharmaceutical composition comprising a RIPK2 inhibitor. Any of the RIPK2 inhibitors and pharmaceutical compositions comprising the RIPK2 inhibitor as described herein can be used. For example, useful RIPK2 inhibitors include those that can inhibit the activity of RIPK2 and/or its expression. In some embodiments, the RIPK2 inhibitors can be selective inhibitors over other RIP kinases such as RIPK1 and/or RIPK3, for example, with a selectivity of about 2-fold, about 4-fold, about 10-fold, or higher. In some embodiments, the RIPK2 inhibitor has substantially no activity against other RIP kinases. However, in some embodiments, the RIPK2 inhibitor can also be a dual or multi RIP kinases inhibitor, or a pan-RIP kinase inhibitor. In some embodiments, the RIPK2 inhibitor is a small molecule RIPK2 inhibitor described herein.

In some embodiments, the present invention also provides a method of protecting neuronal cells in a subject comprising administering to the subject an effective amount of a RIPK2 inhibitor or a pharmaceutical composition comprising a RIPK2 inhibitor. In some embodiments, the method protects neuronal cells from neuroinflammation and/or toxicity from gliosis (activation of microglia and/or astrocytes), for example, mediated by an abnormal protein such as α-synuclein, amyloid-β, and/or tau. In some embodiments, the subject suffers from one or more neurodegenerative diseases or disorders (e.g., any of those described herein), for example, Parkinson's disease or Alzheimer's disease. Any of the RIPK2 inhibitors and pharmaceutical compositions comprising the RIPK2 inhibitor as described herein can be used. For example, useful RIPK2 inhibitors include those that can inhibit the activity of RIPK2 and/or its expression. In some embodiments, the RIPK2 inhibitors can be selective inhibitors over other RIP kinases such as RIPK1 and/or RIPK3, for example, with a selectivity of about 2-fold, about 3-fold, about 4-fold, about 5-fold, about 10-fold, or higher. In some embodiments, the RIPK2 inhibitor has substantially no activity against other RIP kinases. However, in some embodiments, the RIPK2 inhibitor can also be a dual or multi RIP kinases inhibitor, or a pan-RIP kinase inhibitor. In some embodiments, the RIPK2 inhibitor is a small molecule RIPK2 inhibitor described herein.

In any of the methods described herein, the RIPK2 inhibitor can be formulated for administration and/or administered to a subject (e.g., human) via an intended route of administration. For example, in some embodiments, the RIPK2 inhibitor can be administered intravenously, subcutaneously, intra-arterially, intraperitoneally, ophthalmically, intramuscularly, buccally, rectally, vaginally, intraorbitally, intracerebrally, intradermally, intracranially, intraspinally, intraventricularly, intrathecally, intracisternally, intracapsularly, intrapulmonary, intranasally, transmucosally, transdermally, and/or via inhalation. In some specific embodiments, the RIPK2 inhibitor can be administered via oral administration. In some embodiments, the RIPK2 inhibitor can be administered via parenteral administration (e.g., injection such as intravenous injection). Typically, the RIPK2 inhibitor is administered in an amount effective in inhibiting one or more activities selected from NOD1-dependent activation of NFκB, NOD2-dependent activation of NF-kB, microglial activation, and reactive astrocytes formation.

In certain embodiments, the RIPK2 inhibitors (e.g., small molecule inhibitors) described herein can be administered in combination with at least one other therapeutically active agent. The two or more agents can be co-administered, co-formulated, administered separately, or administered sequentially. For example, in some embodiments, the method is for treating Parkinson's disease and the RIPK2 inhibitor can be administered in combination with levodopa, carbodopa or a combination thereof, pramipexole, ropinirole, rotigotine, selegiline, rasagiline, entacapone, tolcapone, benztropine, trihexyphenidyl, or amantadine, or a pharmaceutically acceptable salt thereof. In some embodiments, the method is for treating Alzheimer's disease and the RIPK2 inhibitor can be administered in combination with donepezil, galantamine, memantine, rivastigmine, anti-Abeta (amyloid beta) therapies including aducanumab, crenezumab, solanezumab, and gantenerumab, small molecule inhibitors of BACE1 including verubecestat, AZD3293 (LY3314814), elenbecestat (E2609), LY2886721, PF-05297909, JNJ-54861911, TAK-070, VTP-37948, HPP854, CTS-21166, or anti-tau therapies such as LM™ (leuco-methylthioninium-bis (hydromethanesulfonate)), or a pharmaceutically acceptable salt thereof.

In some embodiments, the RIPK2 inhibitor can be administered in combination with inhibitors of other RIP kinases, such as RIPK1, RIPK3, RIPK4, and/or RIPK5. For example, in some embodiments, the RIPK2 inhibitor can be administered in combination with a RIPK1 inhibitor. Suitable RIPK1 inhibitors include those known in the art, for example, those described in U.S. Pat. No. 9,896,458 and WO2017/096301, the content of which is herein incorporated by reference in its entirety.

Certain embodiments include a method of inhibiting activation of CNS resident innate immune cells. In some embodiments, the method comprises contacting the immune cells with an effective amount of a RIPK2 inhibitor (e.g., described herein). In some embodiments, the method inhibits activation of CNS resident innate immune cells mediated by an abnormal protein, such as abnormally aggregated proteins, e.g., α-synuclein, amyloid-β, and/or tau. In some embodiments, the contacting can be in vivo. In some embodiments, when needed, the in vivo activation of CNS resident innate immune cells or the inhibition thereof can be measured by various imaging methods. For example, Dipont A. C. et al. described a Translocator Protein-18 kDa (TSPO) Positron Emission Tomography (PET) imaging method for detecting activated microglia in neurodegenerative diseases. Intl. J. Mol. Sci., 18(4):785 (2017). For example, in some embodiments, the contacting occurs in the CNS of a subject having one or more neurodegenerative disease (e.g., any of those described herein, such as Parkinson's disease or Alzheimer's disease). In some embodiments, the contacting can be in vitro. In some embodiments, the contacting can also be ex vivo. In some embodiments, the amount of RIPK2 inhibitor is effective to reduce the level of one or more inflammatory or neurotoxic mediators secreted by the CNS resident innate immune cells compared to a control (e.g., substantially same cells that are treated/contacted with a placebo without RIPK2 inhibitor). For example, in some embodiments, the contacting with RIPK2 inhibitor can be effective in reducing the level of TNFα, IL-1α, IL-1β, C1q, IL-6, or a combination thereof, compared to a control.

### Kits

In certain embodiments, a kit for the treatment of a neurodegenerative disease or disorder thereof, comprises a pharmaceutical composition of at least one RIPK2 inhibitor and a pharmaceutically acceptable carrier, excipient or diluent. In some embodiments, a kit can further comprise a label with instructions for methods of treatment or administration. In certain embodiments, the kit further comprises at least one additional therapeutically active compound (e.g., as described herein).

Two or more inhibitors of RIPK2 can be included in the kit, which can comprise small molecules, siRNAs, shRNAs, micro RNAs, antibodies, aptamers, enzymes, a gene editing system, hormones, inorganic compounds, oligonucleotides, organic compounds, polynucleotides, peptides, or synthetic compounds.

### EXAMPLES

### Example 1: p-RIPK2 is Elevated in the SNpc of Human PD Postmortem Tissues

Study Rationale and Objectives:

The aim of this study was to investigate expressions of phosphorylated RIPK2 (p-RIPK2), RIPK2 and NOD2 in post-mortem human brain tissues of patients with PD and investigate if NOD2, pattern recognition receptor, can be the receptor for α-synuclein aggregates in microglia in PD. Human post-mortem tissue samples (substantia nigra, SN) from neurologically unimpaired subjects with normal (n=4) and from subjects with PD (n=7) were obtained from Division of Neuropathology, Department of Pathology of Johns Hopkins University. Diagnosis of PD was confirmed by pathological and clinical criteria. p-RIPK2, RIPK2 and NOD2 levels were monitored in human post-mortem substantia nigra (SN) brain tissue from PD patients and controls by immunostaining, PLA, real-time PCR and Western blot analyses. METHODS

Immunohistochemistry (IHC) for PD Postmortem Brain:

Slides with 10-μm thickness of formalin-fixed paraffin-embedded human postmortem SN tissues were obtained from the Division of Neuropathology, Department of Pathology, Johns Hopkins University. The tissue sections were deparaffinized and rehydrated, and then heat-induced epitope retrieval was performed with citrate-based antigen unmasking solutions (Vector Laboratories). Then, the slides were stained with rabbit polyclonal p-RIPK2 or Iba-1 antibody. All sections were stained with H&E.

In Situ Proximity Ligation Assay (PLA):

The tissue sections were used for in situ proximity ligation assay (Sigma) following manufacturer's instruction. Briefly, sections were blocked with a provided blocking buffer and incubated with primary antibodies at 4° C. for 12 hours. The Minus or Plus probe conjugated secondary antibodies were then added and incubated at 37° C. for 1 hour. After incubation, the ligation mix was added to each coverslip and incubated at 37° C. for another 30 min. The signals were then amplified by addition of amplification-polymerase containing reaction solution. The coverslips were mounted after hematoxylin counter staining.

Real-Time RT-PCR (qPCR):

The total RNA was isolated from the human SN post-mortem tissues and the mouse ventral midbrain tissues using RNeasy® Plus Micro Kit (Qiagen). The first-strand cDNA was then synthesized with SuperScript® IV First-Strand Synthesis System (Invitrogen). The real-time PCR was performed with the SYBR Green reagent by a ViiA™ 7 real-time PCR system. The 2^{-ΔΔCT} method (Livak and Schmittgen, Methods 25:402-8 (2001)) was used for calculating the values. All ACT values were normalized to GAPDH.

Western Blot Analysis:

The post-mortem tissues of the human SN were homogenized in the tissue lysis buffer containing 150 mM NaCl, 5 mM EDTA, 10 mM Tris-HCl pH 7.4, Nonidet P-40, 10 mM Na-β-glycerophosphate, complete protease inhibitor cocktail (Roche), and phosphatase inhibitor cocktail I and II (Sigma-Aldrich) as previously described (Ko et al., Proc. Natl. Acad. Sci. USA 107:16691-6 (2010)). The lysates were then utilized to dilute in 2× Laemmli buffer (Bio-Rad). The 20 μg of proteins were resolved with 8-16% gradient SDS-PAGE gels and transferred to nitrocellulose membranes. The nitrocellulose membrane was blocked with 5% non-fat dry milk in 0.1% Tween-20 containing Tris-buffered saline for 1 hours at RT. The membrane was then incubated with primary antibodies as follows: anti-NOD2, anti-RIPK2, and anti-pRIPK2 antibodies at 4° C. for overnight. After three times of washing, the membranes were incubated with HRP-conjugated rabbit or mouse secondary antibodies (GE Healthcare) for 1 hour at RT. The signals were utilized to visualize by chemiluminescence reagents (Thermo Scientific). The membranes were then re-probed with HRP-conjugated β-actin antibody (Sigma).

Results:

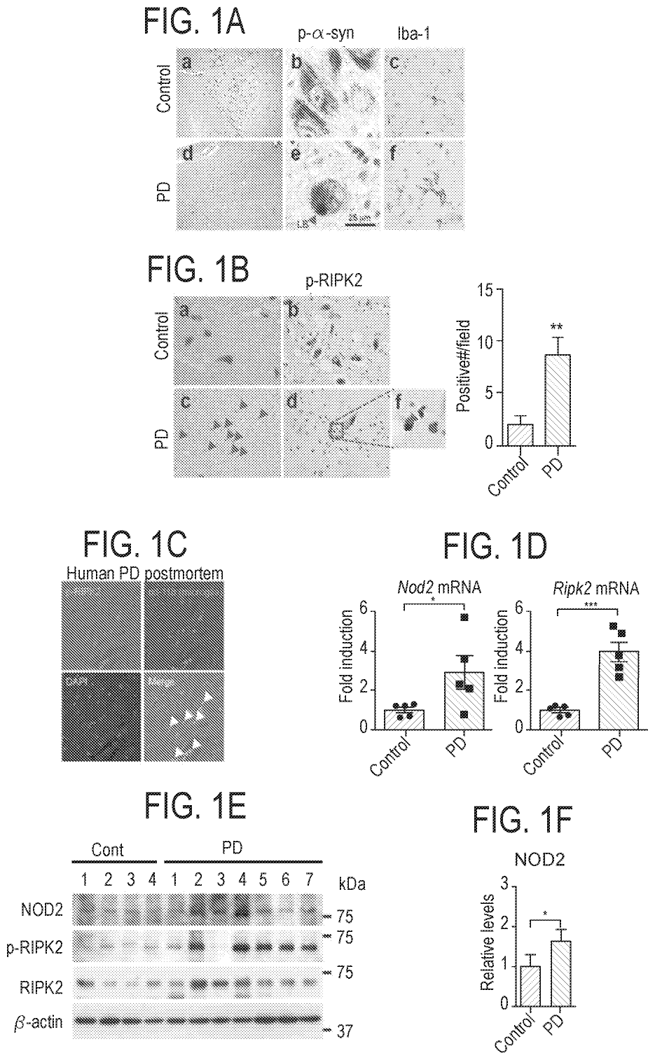

Our data indicates that p-RIPK2 immunoreactivity is significantly increased in the SN (FIG. 1B) of PD patient samples with a robust microglia activation and lewy body (LB) pathology (FIG. 1A) and the p-RIPK2 signals are mainly co-localized with cd-11b positive microglia in the SN of PD patient samples as assessed by immunohistochemistry (FIG. 1C). NOD2 and RIPK2 mRNA levels are significantly increased in the SN from PD patient samples as assessed by qPCR analysis (FIG. 1D). Also, NOD2, RIPK2 and p-RIPK2 protein levels are significantly increased in the SN from PD patient samples as assessed by Western blot analysis (FIG. 1E-G). Taken together, these data indicate that the site of activation of RIPK2 is predominantly microglia in PD brains and excessive RIPK2 activation plays a pivotal role in the pathogenesis of PD.

To ascertain whether NOD2, pattern recognition receptor, can be the receptor for α-synuclein aggregates in microglia in PD, we performed in situ Duolink proximity ligation assay (PLA), a powerful technology capable of detecting single protein events such as protein-protein interactions both in vitro and in vivo. We observed a number of strong positive signals (FIG. 1H) in the presence of specific antibodies for α-synuclein aggregates and NOD2 in the SN of PD post-mortem, suggesting the interaction between α-synuclein aggregates and NOD2 in microglia (FIG. 1H). This data indicates that α-synuclein is the ligand for NOD2 receptor.

TABLE 1
mRNA levels (relative fold) of NOD2 and
RIPK2 (related to FIG. 1D). The values are the
mean ± S.E.M., n = 5. (*P <0.05, *** P <0.001).

| Mrna | Control | PD |
|---|---|---|
| NOD2 | 1 ± 0.12 | 2.90 ± 0.83* |
| RIPK2 | 1 ± 0.11 | 3.98 ± 0.49*** |

TABLE 2
Relative protein levels of NOD2 (related to FIG. 1F).
The values are the mean ± S.E.M.,
n = 4 (control), n = 7 (PD). (*P <0.05).

| Protein | Control | PD |
|---|---|---|
| NOD2 | 1.00 ± 0.16 | 1.63 ± 0.15* |

TABLE 3
Relative protein levels of p-RIPK2 and RIPK2 (related to FIG. 1G).
The values are the mean ± S.E.M., n = 4 (control),
n = 7 (PD). (**P <0.01, *** P <0.001).

| Protein | Control | PD |
|---|---|---|
| p-RIPK2 | 1 ± 0.19 | 5.02 ± 0.79*** |
| RIPK2 | 1 ± 0.23 | 2.77 ± 0.37** |

### Example 2: α-Synuclein PFFs-Activated Microglia Induce RIPK2, NOD1 and NOD2 In Vitro

Study Rationale and Objectives:

The aim of this study was to investigate whether α-synuclein PFFs induce mRNA expression of RIPK2, NOD1 and NOD2 in primary microglia by qPCR analysis.

### Methods

Comparative qPCR:

The total RNA from cultured cells was extracted with RNA isolation kit (Qiagen, CA) following the instruction provided by the company. RNA concentration was measured spectrophotometrically using NanoDrop 2000 (Biotek, Winooski, Vt.). 1-2 μg of the total RNA were reverse-transcribed to cDNA using the High-Capacity cDNA Reverse Transcription System (Life Technologies, Grand Island, N.Y.). Comparative qPCR was performed in duplicate or triplicate for each sample using fast SYBR Green Master Mix (Life Technologies) and ViiA 7 Real-Time PCR System (Applied Biosystems, Foster City, Calif.). The expression levels of targeted genes were normalized to the expression of β-actin and calculated based on the comparative cycle threshold Ct method (2-ΔΔCt).

Results:

We obtained a total >600 differently expressed genes from RNAseq analysis using primary microglia treated with endotoxin free α-synuclein PFFs. Among them, NOD2 and RIPK2 were top-ranked. We confirmed that the mRNA levels of RIPK2 and NOD2 are significantly increased in α-synuclein PFFs-activated microglia, thus can be therapeutic targets for neurodegenerative disorders associated with activated microglia in brain.

TABLE 4
mRNA levels (relative fold) of RIIPK2, NOD1 and NOD2 in normal
(PBS) and α-synuclein PFFs activated mouse primary microglia.
The values are the mean ± SEM, n = 3. (**P <0.01, *** P <0.001).

| Mrna | PBS | α-synuclein PFFs |
|---|---|---|
| RIPK2 | 1 ± 0.14 | 38.75 ± 2.81*** |
| NOD1 | 1 ± 0.16 | 2.56 ± 0.27** |
| NOD2 | 1 ± 0.13 | 19.33 ± 1.82*** |

### Example 3. Depletion of NOD2 or RIPK2 Suppress α-Synuclein PFFs Induced Microglia Activation and A1 Reactive Astrocytes

Study Rationale and Objectives:

The aim of this study was to 1) assess the depletion effect of NOD2 or RIPK2 on cytokine production such as TNFα, IL-1α and complement C1q (A1 astrocyte inducers) by primary microglia activated with α-synuclein PFFs, 2) investigate the depletion effect of NOD2 or RIPK2 on the differentiation of neurotoxic and reactive A1 astrocytes induced by activated microglia, and 3) investigate the depletion effect of NOD2 or RIPK2 on the reactive A1 astrocytes induced neuronal toxicity. To this end, qPCR and neuronal toxicity assays were employed.

### Methods

α-synuclein purification and α-synuclein PFFs preparation: Recombinant mouse α-synuclein proteins were purified as previously described with an IPTG-independent inducible pRK172 vector system (Nat. Protoc. 9:2135-46 (2014))). Endotoxin was depleted by ToxinEraser endotoxin removal kit (Genscript, NJ, USA). α-synuclein PFFs (5 mg ml⁻¹) was prepared in PBS while stirring with a magnetic stirrer (1,000 rpm at 37° C.). After a week of incubation of the α-synuclein protein, aggregates were diluted to 0.1 mg ml−1 with PBS and sonicated for 30 s (0.5 s pulse on/off) at 10% amplitude (Branson Digital sonifier, Danbury, Conn., USA). α-synuclein PFFs was validated using atomic force microscopy and transmission electron microscopy, and the ability to induce phospho-serine 129 α-synuclein (p-α-synSer129) was confirmed using immunostaining. α-synuclein PFFs was stored at −80° C. until use.

Primary Neuron, Microglia and Astrocyte Cell Cultures, and α-Synuclein PFFs Treatment:

NOD2 or RIPK2 knockout mice was obtained from Jackson Laboratories (Bar Harbor, Me., USA). Primary cortical neurons were prepared from embryonic day 15.5 pups and cultured in Neurobasal medium (Gibco) supplemented with B-27, 0.5 mM L-glutamine, penicillin and streptomycin (Invitrogen, Grand Island, N.Y., USA) on tissue-culture plates coated with poly-L-lysine. The neurons were maintained by changing the medium every 3-4 days. Primary microglial and astrocyte cultures were performed as described previously (PMID: 26157004). Whole brains from mouse pups at postnatal day 1 (P1) were obtained. After removal of the meninges, the brains were washed in DMEM/F12 (Gibco) supplemented with 10% heat-inactivated FBS, 50 U ml⁻¹ penicillin, 50 μg ml⁻¹ streptomycin, 2 mM L-glutamine, 100 μM non-essential amino acids and 2 mM sodium pyruvate (DMEM/F12 complete medium) three times. The brains were transferred to 0.25% trypsin-EDTA followed by 10 min of gentle agitation. DMEM/F12 complete medium was used to stop the trypsinization. The brains were washed three times in this medium again. A single-cell suspension was obtained by trituration. Cell debris and aggregates were removed by passing the single-cell suspension through a 100-μm nylon mesh. The final single-cell suspension thus achieved was cultured in T75 flasks for 13 days, with a complete medium change on day 6. The mixed glial cell population was separated into astrocyte-rich and microglia-rich fractions using the EasySep Mouse CD11b Positive Selection Kit (StemCell). The magnetically separated fraction containing microglia and the pour-off fraction containing astrocytes were cultured separately.

Microglia prepared from wild type (WT), NOD2 knockout (KO), RIPK2 KO mice were treated with and α-synuclein PFF (final concentration 1 μg/mL) for 30 min followed by qPCR assay.

The conditioned medium from the primary wild type microglia (WT PFFs-MCM), NOD2 knockout microglia (NOD2^{-/-} PFF-MCM), or RIPK2 knockout microglia (RIPK2^{-/-} PFFs-MCM) treated with α-synuclein PFFs were collected and applied to primary astrocytes for 24 h. The conditioned medium from activated astrocytes by 1) WT PFFs-MCM, which we define as α-syn PFF-ACM, 2) by NOD2^{-/-} PFFs-MCM, which we define as NOD2^{-/-} PFFs-ACM, 3) by RIPK2^{-/-} PFFs-MCM, which we define as RIPK2^{-/-} PFF-ACM, were collected with complete, Mini, EDTA-free Protease Inhibitor Cocktail (Sigma) and concentrated with Amicon Ultra-15 centrifugal filter unit (10 kDa cutoff) (Millipore) until approximately 50× concentrated. The total protein concentration was determined using Pierce BCA protein assay kit (Thermo Scientific), and 15 or 50 μg ml^{-/-} of total protein was added to mouse primary neurons for the neuronal cell death assay.

Comparative qPCR:

The total RNA from cultured cells was extracted with RNA isolation kit (Qiagen, CA) following the instruction provided by the company. RNA concentration was measured spectrophotometrically using NanoDrop 2000 (Biotek, Winooski, Vt.). 1-2 μg of the total RNA were reverse-transcribed to cDNA using the High-Capacity cDNA Reverse Transcription System (Life Technologies, Grand Island, N.Y.). Comparative qPCR was performed in duplicate or triplicate for each sample using fast SYBR Green Master Mix (Life Technologies) and ViiA 7 Real-Time PCR System (Applied Biosystems, Foster City, Calif.). The expression levels of targeted genes were normalized to the expression of β-actin and calculated based on the comparative cycle threshold Ct method (2-ΔΔCt).

Cell Viability by LDH and Alamar Blue Assays:

Primary cultured cortical neurons were treated with PFF-ACM or NOD2^{-/-}-PFF-ACM or RIPK2^{-/-}-PFF-ACM for 24 hr. Cell viability was determined by two methods: The AlamarBlue (Invitrogen) and LDH assay (Sigma). Cell death was assessed through AlamarBlue assay, according to the manufacturer's protocol. LDH activity in culture medium, representing relative cell viability and membrane integrity, was measured using the LDH assay kit spectrophotometrically, following the manufacturer's instructions. Triplicate wells were assayed for each condition.

Results:

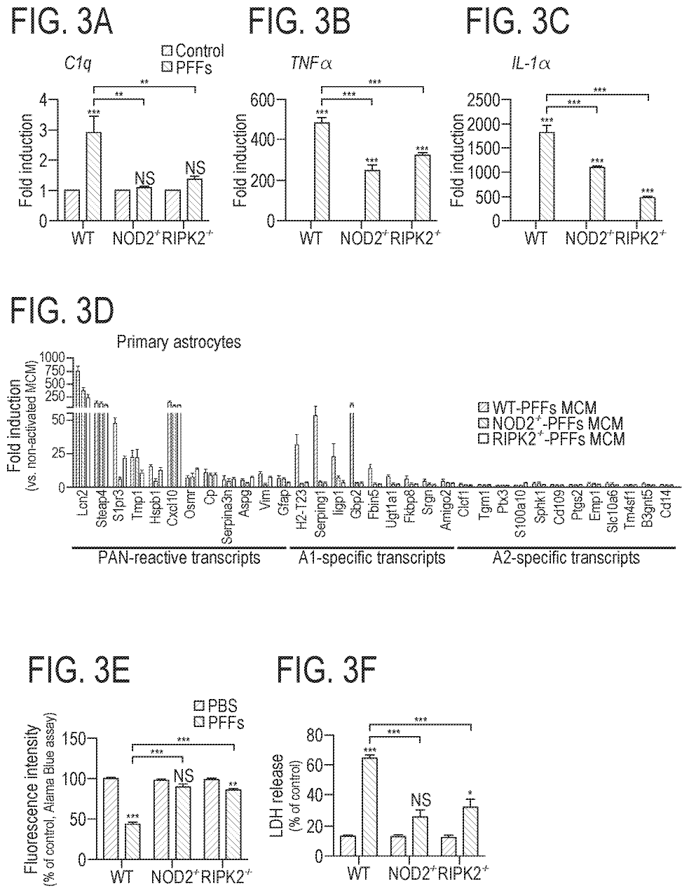

Our data indicates that α-synuclein PFFs can induce TNFα, IL-1α, and C1q, known as reactive A1 astrocyte inducers, in microglia (FIGS. 3A, 3B, and 3C) and covert A1 astrocytes (FIG. 3D). Importantly, depletion of NOD2 or RIPK2 in microglia suppresses the release of A1 astrocyte inducer from microglia ((FIGS. 3A, 3B, and 3C) and subsequent A1 astrocyte conversion (FIG. 3D). The α-synuclein PFFs-induced A1 astrocyte-conditioned medium (PFF-ACM) is toxic to primary cortical neurons, while NOD2^{-/-} or RIPK2^{-/-}-PFF-ACM are significantly less toxic (FIGS. 3E and 3F). This result clearly indicate that inhibition of RIPK2 and/or NOD2 activity blocks the activation of microglia and the formation of neurotoxic A1 astrocyte formation; thus protects neurons.

TABLE 5
mRNA levels (relative fold) of C1q (related to FIG. 3A).
The values are the mean ± SEM, n = 3. (***P <0.001).

| Mrna | Control | PFFs |
|---|---|---|
| WT | 1 ± 0.02 | 2.91 ± 0.55*** |
| NOD2^{-/-} | 1 ± 0.03 | 1.10 ± 0.02 ^{NS} |
| RIPK2^{-/-} | 1 ± 0.01 | 1.37 ± 0.10 ^{NS} |

TABLE 6
mRNA levels (relative fold) of TNFα (related to FIG. 3B).
The values are the mean ± SEM, n = 3. (***P <0.001).

| Mrna | Control | PFFs |
|---|---|---|
| WT | 1 ± 0.21 | 483.69 ± 23.85*** |
| NOD2^{-/-} | 1 ± 0.18 | 247.68 ± 27.12*** |
| RIPK2^{-/-} | 1 ± 0.14 | 326.05 ± 10.45*** |

TABLE 7
mRNA levels (relative fold) of IL-1α (related to FIG. 3C).
The values are the mean ± SEM, n = 3. (***P <0.001).

| Mrna | Control | PFFs |
|---|---|---|
| WT | 1 ± 0.13 | 1831.49 ± 137.34*** |
| NOD2^{-/-} | 1 ± 0.18 | 1097.87 ± 25.48*** |
| RIPK2^{-/-} | 1 ± 0.15 | 473.40 ± 20.25*** |

TABLE 8
Fluorescence intensity (% of control; related to FIG. 3E).
The values are the mean ± SEM,
n = 3. (**P <0.01, ***P <0.001).

| Intensity | PBS control | PFFs |
|---|---|---|
| WT | 100.00 ± 1.16 | 43.33 ± 1.45*** |
| NOD2^{-/-} | 97.67 ± 0.88 | 89.04 ± 3.61^{NS} |
| RIPK2^{-/-} | 98.67 ± 1.45 | 85.67 ± 1.48** |

TABLE 9
LDH release (% of positive control; related to FIG. 3F).
The values are the mean ± SEM,
n = 3. (*P <0. 05, ***P <0.001).

| % of positive control | PBS control | PFFs |
|---|---|---|
| WT | 13.02 ± 0.58 | 64.33 ± 2.03*** |
| NOD2^{-/-} | 12.66 ± 2.23 | 25.67 ± 4.81^{NS} |
| RIPK2^{-/-} | 12.32 ± 1.20 | 31.14 ± 5.51* |

### Example 4. Depletion of NOD2 or RIPK2 Suppress α-Synuclein PFFs Induced Microglia Morphological Changes and Migration

Study Rationale and Objectives:

The aim of this study was to 1) assess the depletion effect of NOD2 or RIPK2 on morphological changes and migration induced by α-synuclein PFFs. To explore this, morphology assay, qPCR and migration assay were employed.

### Methods

Morphological Assay:

The primary cultured microglia were plated onto poly-D-lysine-coated 12 well-plate. After 12 hours of α-synuclein PFFs treatment, the morphologically changed amoeboid form of microglia were counted. The cells were counterstained with DAPI.

Comparative Quantitative Real Time PCR (qPCR):

The total RNA from cultured cells was extracted with RNA isolation kit (Qiagen, CA) following the instruction provided by the company. RNA concentration was measured spectrophotometrically using NanoDrop 2000 (Biotek, Winooski, Vt.). 1-2 μg of the total RNA were reverse-transcribed to cDNA using the High-Capacity cDNA Reverse Transcription System (Life Technologies, Grand Island, N.Y.). Comparative qPCR was performed in duplicate or triplicate for each sample using fast SYBR Green Master Mix (Life Technologies) and ViiA 7 Real-Time PCR System (Applied Biosystems, Foster City, Calif.). The expression levels of targeted genes were normalized to the expression of β-actin and calculated based on the comparative cycle threshold Ct method (2-ΔΔCt).

Migration Assay:

For in vitro cell migration assay, primary cultured microglia were seeded onto poly-D-lysine-coated 12-well polycarbonate cell culture inserts and bottom of culture dishes. After 12 hours of α-syn PFFs treatment in the culture dishes, the migrated microglia on the bottom side of inserts were stained with Iba-1 antibody. The migrate index were then calculated through the ratio between the number of Iba-1 positive migrated microglia with respect to PBS control.

Results:

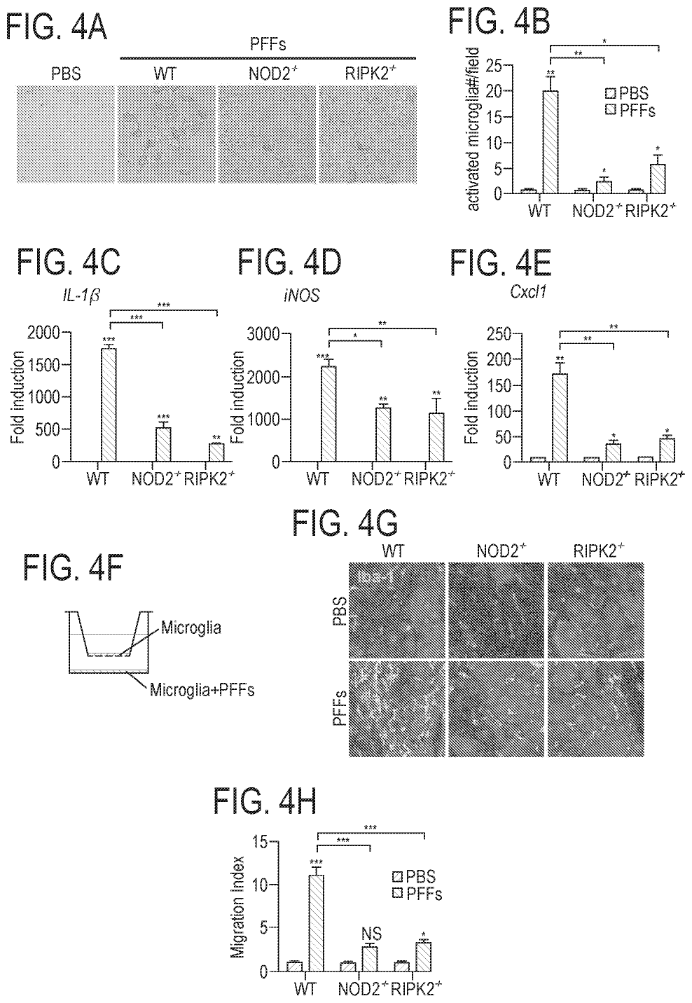

Our data indicates that α-synuclein PFF significantly induce microglia morphological change. Deletion of NOD2 or RIPK2 in microglia suppresses the amoeboid form of microglia (FIGS. 4A and 4B). The mRNA expression of PFFs-induced pro-inflammatory genes such as IL-la and iNOS were dramatically reduced in NOD2^{-/-} or RIPK2^{-/-} microglia (FIGS. 4C and 4D). The migration ability and chemokine Cxcl1 expression also reduced in NOD2^{-/-} and RIPK2^{-/-} microglia (FIGS. 4E, 4F, 4G, and 4H).

TABLE 10
The morphological changed microglia
(the number of changed microglia; related to FIG. 4B).
The values are the mean ± SEM, n = 3. (*P <0. 05, ***P <0.001).

| # of changed cells | PBS control | PFFs |
|---|---|---|
| WT | 1.00 ± 0.13 | 18.97 ± 3.82** |
| NOD2^{-/-} | 0.93 ± 0.15 | 3.14 ± 0.97* |
| RIPK2^{-/-} | 0.97 ± 0.11 | 6.52 ± 1.71* |

TABLE 11
mRNA levels (relative fold) of IL-1α (related to FIG. 4C).
The values are the mean ± SEM, n = 3. (**P <0. 01, ***P <0.001).

| % of positive control | PBS control | PFFs |
|---|---|---|
| WT | 1.00 ± 0.14 | 1750.70 ± 62.83*** |
| NOD2^{-/-} | 1.00 ± 0.16 | 527.69 ± 81.76*** |
| RIPK2^{-/-} | 1.00 ± 0.32 | 267.74 ± 10.08** |

TABLE 12
mRNA levels (relative fold) of iNOS (related to FIG. 4D).
The values are the mean ± SEM, n = 3. (**P <0.01, ***P <0.001).

| % of positive control | PBS control | PFFs |
|---|---|---|
| WT | 1.00 ± 0.19 | 2219.47 ± 178.31*** |
| NOD2^{-/-} | 1.00 ± 0.13 | 1279.06 ± 70.83** |
| RIPK2^{-/-} | 1.00 ± 0.42 | 1144.39 ± 339.95** |

TABLE 13
mRNA levels (relative fold) of Cxcl1 (related to FIG. 4E).
The values are the mean ± SEM, n = 3. (*P < 0.05, **P < 0.01).

| % of positive control | PBS control | PFFs |
|---|---|---|
| WT | 1.00 ± 0.18 | 170.03 ± 22.86** |
| NOD2^{-/-} | 1.00 ± 0.09 | 3.55 ± 0.77* |
| RIPK2^{-/-} | 1.00 ± 0.08 | 4.58 ± 0.74* |

TABLE 14
Migration index of microglia (related to FIG. 4C).
The values are the mean ± SEM, n = 3.
(*P < 0.05, **P < 0.01).

| Migration index | PBS control | PFFs |
|---|---|---|
| WT | 1.00 ± 0.11 | 11.04 ± 1.72*** |
| NOD2^{-/-} | 0.97 ± 0.14 | 3.41 ± 0.59^{NS} |
| RIPK2^{-/-} | 0.98 ± 0.16 | 3.97 ± 0.22* |

### Example 5: Inhibitors of RIPK2 Suppress α-Synuclein PFFs Induced Microglia Activation and A1 Reactive Astrocytes In Vitro

Study rationale and objectives: The object of this study was to 1) assess the effect of RIPK2 inhibitors on cytokine production such as TNFα, IL-1α and complement C1q (reactive A1 astrocyte inducers) by primary microglia activated with α-synuclein PFFs, 2) investigate the effect of RIPK2 inhibitors on the formation of A1 neurotoxic astrocytes induced by activated microglia, and 3) investigate the effect of RIPK2 inhibitors on the reactive A1 astrocytes induced neuronal toxicity. To this end, qPCR and neuronal toxicity assays were employed.

### Methods

α-Synuclein Purification and α-Synuclein PFFs Preparation:

Recombinant mouse α-synuclein proteins were purified as previously described with an IPTG-independent inducible pRK172 vector system (Nat Protoc. 9(9):2135-46 (2014)). Endotoxin was depleted by ToxinEraser endotoxin removal kit (Genscript, NJ, USA). α-synuclein PFFs (5 mg ml⁻¹) was prepared in PBS while stirring with a magnetic stirrer (1,000 rpm at 37° C.). After a week of incubation of the α-synuclein protein, aggregates were diluted to 0.1 mg ml⁻¹ with PBS and sonicated for 30 s (0.5 s pulse on/off) at 10% amplitude (Branson Digital sonifier, Danbury, Conn., USA). α-synuclein PFFs was validated using atomic force microscopy and transmission electron microscopy, and the ability to induce phospho-serine 129 α-synuclein (p-α-synSer129) was confirmed using immunostaining. α-synuclein PFFs was stored at −80° C. until use.

Primary Neuron, Microglia and Astrocyte Cell Cultures, and α-Synuclein PFFs Treatment:

NOD2 or RIPK2 knockout mice was obtained from Jackson Laboratories (Bar Harbor, Me., USA). Primary cortical neurons were prepared from embryonic day 15.5 pups and cultured in Neurobasal medium (Gibco) supplemented with B-27, 0.5 mM L-glutamine, penicillin and streptomycin (Invitrogen, Grand Island, N.Y., USA) on tissue-culture plates coated with poly-L-lysine. The neurons were maintained by changing the medium every 3-4 days. Primary microglial and astrocyte cultures were performed as described previously (PMID: 26157004). Whole brains from mouse pups at postnatal day 1 (P1) were obtained. After removal of the meninges, the brains were washed in DMEM/F12 (Gibco) supplemented with 10% heat-inactivated FBS, 50 U ml⁻¹ penicillin, 50 μg ml⁻¹ streptomycin, 2 mM L-glutamine, 100 μM non-essential amino acids and 2 mM sodium pyruvate (DMEM/F12 complete medium) three times. The brains were transferred to 0.25% trypsin-EDTA followed by 10 min of gentle agitation. DMEM/F12 complete medium was used to stop the trypsinization. The brains were washed three times in this medium again. A single-cell suspension was obtained by trituration. Cell debris and aggregates were removed by passing the single-cell suspension through a 100-μm nylon mesh. The final single-cell suspension thus achieved was cultured in T75 flasks for 13 days, with a complete medium change on day 6. The mixed glial cell population was separated into astrocyte-rich and microglia-rich fractions using the EasySep Mouse CD11b Positive Selection Kit (StemCell). The magnetically separated fraction containing microglia and the pour-off fraction containing astrocytes were cultured separately.

Gefitinib or GSK583 (10 μM) was added to microglia prepared from WT, NOD2 KO, or RIPK2 KO for 30 min and α-synuclein PFFs (final concentration 1 μg/mL) was further incubated for 4 h followed by qPCR.

The conditioned medium from the primary wild type microglia (PFF-MCM), Gefitinib treated microglia (PFF-gefitinib-MCM), or GSK583 treated microglia (PFF-GSK583-MCM) treated with α-synuclein PFF were collected and applied to primary astrocytes for 24 h. The conditioned medium from activated astrocytes by 1) PFF-MCM, which we define as PFF-ACM, 2) by PFF-gefitinib-MCM, which we define as PFF-gefitinib-ACM, 3) by PFF-GSK583-MCM, which we define as PFF-GSK583-ACM, were collected with complete, Mini, EDTA-free Protease Inhibitor Cocktail (Sigma) and concentrated with Amicon Ultra-15 centrifugal filter unit (10 kDa cutoff) (Millipore) until approximately 50× concentrated. The total protein concentration was determined using Pierce BCA protein assay kit (Thermo Scientific), and 15 or 50 μg ml⁻¹ of total protein was added to mouse primary neurons for the neuronal cell death assay.

Comparative qPCR:

The total RNA from cultured cells was extracted with RNA isolation kit (Qiagen, CA) following the instruction provided by the company. RNA concentration was measured spectrophotometrically using NanoDrop 2000 (Biotek, Winooski, Vt.). 1-2 μg of the total RNA were reverse-transcribed to cDNA using the High-Capacity cDNA Reverse Transcription System (Life Technologies, Grand Island, N.Y.). Comparative qPCR was performed in duplicate or triplicate for each sample using fast SYBR Green Master Mix (Life Technologies) and ViiA 7 Real-Time PCR System (Applied Biosystems, Foster City, Calif.). The expression levels of targeted genes were normalized to the expression of β-actin and calculated based on the comparative cycle threshold Ct method (2-ΔΔCt).

Cell Viability by LDH and Alamar Blue Assays:

Primary cultured cortical neurons were treated with PFF-ACM, PFF-gefitinib-MCM or PFF-GSK583-ACM for 24 hr. Cell viability was determined by two methods: The AlamarBlue (Invitrogen) and LDH assay (Sigma). Cell death was assessed through AlamarBlue assay, according to the manufacturer's protocol. LDH activity in culture medium, representing relative cell viability and membrane integrity, was measured using the LDH assay kit spectrophotometrically, following the manufacturer's instructions. Triplicate wells were assayed for each condition.

Results:

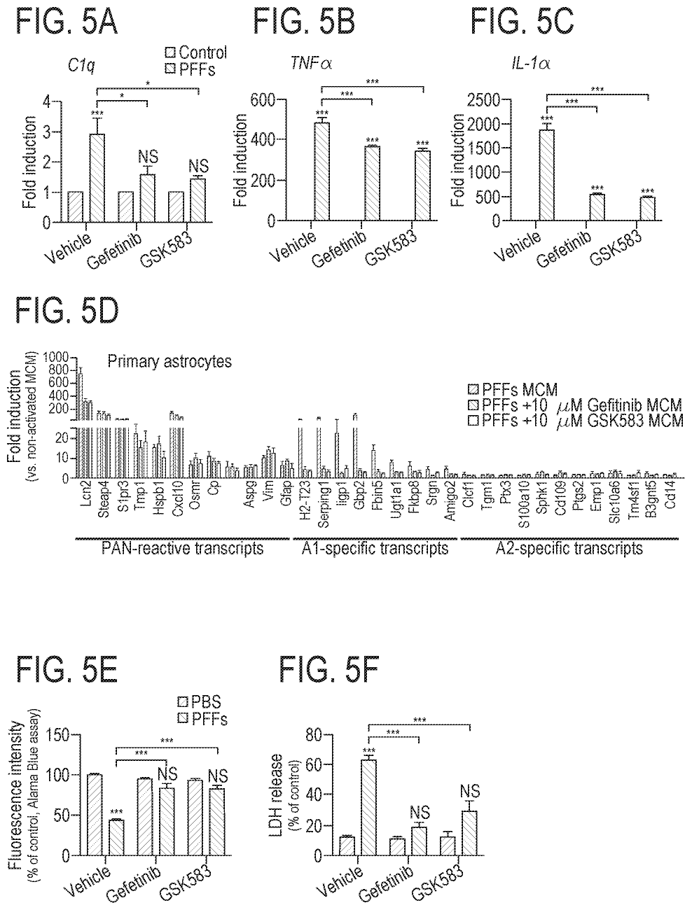

Our data indicates that α-synuclein PFFs induce TNFα, IL-1α, and C1q in microglia (FIGS. 5A, 5B, and 5C) and covert reactive, neurotoxic A1 astrocytes (FIG. 5D). Treatment of RIPK2 inhibitors such as Gefitinib or GSK583 in microglia significantly suppress the release of A1 astrocyte inducer (FIGS. 5A, 5B, and 5C) from microglia and subsequent A1 astrocyte conversion (FIG. 5D). The α-synuclein PFFs-induced A1 astrocyte-conditioned medium (PFF-ACM) is toxic to primary cortical neurons, while treatment of Gefitinib or GSK583 significantly prevented neuronal cell death mainly induced by neurotoxic astrocytes (FIGS. 5E and 5F).

TABLE 15
mRNA levels (relative fold) of C1q (related to sure 5A).
The values are the mean ± SEM, n = 3.
(***P < 0.001).

| mRNA | Control | PFFs |
|---|---|---|
| WT | 1 ± 0.02 | 2.91 ± 0.55*** |
| NOD2^{-/-} | 1 ± 0.03 | 1.58 ± 0.29*** |
| RIPK2^{-/-} | 1 ± 0.02 | 1.44 ± 0.11*** |

TABLE 16
mRNA levels (relative fold) of TNFα (related to FIG. 5B).
The values are the mean ± SEM, n = 3. (***P < 0.001).

| mRNA | Control | PFFs |
|---|---|---|
| WT | 1 ± 0.21 | 483.69 ± 23.85*** |
| NOD2^{-/-} | 1 ± 0.14 | 366.45 ± 6.70*** |
| RIPK2^{-/-} | 1 ± 0.19 | 340.23 ± 16.65*** |

TABLE 17
mRNA levels (relative fold) of IL-1α (related to FIG. 5C).
The values are the mean ± SEM, n = 3.
(***P < 0.001).

| mRNA | Control | PFFs |
|---|---|---|
| WT | 1 ± 0.13 | 1831.49 ± 137.34*** |
| NOD2^{-/-} | 1 ± 0.12 | 504.48 ± 14.87*** |
| RIPK2^{-/-} | 1 ± 0.31 | 452.113 ± 14.334*** |

TABLE 18
Fluorescence intensity (% of control; related to
FIG. 5E). The values are the mean ± SEM,
n = 3. (***P < 0.001).

| Intensity | PBS control | PFFs |
|---|---|---|
| WT | 100.00 ± 1.16 | 43.33 ± 1.45*** |
| NOD2^{-/-} | 95.6 ± 1.20 | 83.67 ± 6.57 ^{NS} |
| RIPK2^{-/-} | 94.33 ± 1.67 | 83.33 ± 4.63 ^{NS} |

TABLE 19
LDH release (% of positive control; related to FIG. 5F).
The values are the mean ± SEM, n = 3.
(***P < 0.001).

| % of positive control | PBS control | PFFs |
|---|---|---|
| WT | 12.31 ± 0.88 | 63.33 ± 3.18*** |
| NOD2^{-/-} | 11.38 ± 1.45 | 19.07 ± 3.01 ^{NS} |
| RIPK2^{-/-} | 12.32 ± 3.48 | 29.33 ± 7.22 ^{NS} |

### Example 6: Depletion of NOD2 or RIPK2 Significantly Ameliorates Lewy Body (LB) Pathology and Suppresses Microglia Activation in α-Synuclein PFFs-Induced PD Animal Model

Study Rationale and Objectives:

The purpose of this study was to investigate the anti-PD efficacy of NOD2 or RIPK2 depletion in α-synuclein PFFs model PD to validate if NOD2 or RIPK2 can be a viable therapeutic target for PD.

### Methods

Mouse strain for stereotaxic α-synuclein PFFs injection: NOD2 or RIPK2 knockout mice were obtained from the Jackson Laboratories (Bar Harbor, Me.). All housing, breeding, and procedures were performed according to the NIH Guide for the Care and Use of Experimental Animals and approved by the Johns Hopkins University Animal Care and Use Committee.

α-Synuclein Protein Purification and PFF Preparation:

Recombinant mouse α-synuclein proteins were purified as previously described with an IPTG-independent inducible pRK172 vector system. Endotoxin was depleted by ToxinEraser endotoxin removal kit (Genscript, NJ, USA). α-synuclein PFFs (5 mg ml⁻¹) was prepared in PBS while stirring with a magnetic stirrer (1,000 rpm at 37° C.). After a week of incubation of the α-synuclein protein, aggregates were diluted to 0.1 mg ml−1 with PBS and sonicated for 30 s (0.5 s pulse on/off) at 10% amplitude (Branson Digital sonifier, Danbury, Conn., USA). α-synuclein PFFs was validated using atomic force microscopy and transmission electron microscopy, and the ability to induce phospho-serine 129 α-synuclein (p-α-synSer129) was confirmed using immunostaining. α-synuclein PFFs was stored at −80° C. until use.

Stereotaxic α-Synuclein PFFs Injection and Immunohistochemistry (IHC):

For stereotaxic injection of α-synuclein PFFs, 3 months old NOD2 KO or RIPK2 KO male and female were anesthetized with xylazene and ketamine. An injection cannula (26.5 gauge) was applied stereotaxically into the striatum (STR) (mediolateral, 2.0 mm from bregma; anteroposterior, 0.2 mm; dorsoventral, 2.6 mm) unilaterally into the right hemisphere. The infusion of 2 μL α-synuclein PFFs (2.5 μg/mL in PBS) or the same volume of PBS was performed at a rate of 0.2 μL per min. After the final dose, the injection cannula was maintained in the STR for additional 5 min for a complete absorption of the α-synuclein PFFs or PBS then slowly removed from the mouse brain. The head skin was closed by suturing and wound healing and recovery were monitored following surgery. For IHC analysis, animals were perfused and fixed intracardially with ice-cold PBS followed by 4% paraformaldehyde at 3 months after striatal α-synuclein PFFs injections. The brain was removed and processed for immunohistochemistry. IHC for pS129-α-synuclein or Iba-1 was performed at 3 months after the unilateral striatal a-synuclein PFFs injections.

Results:

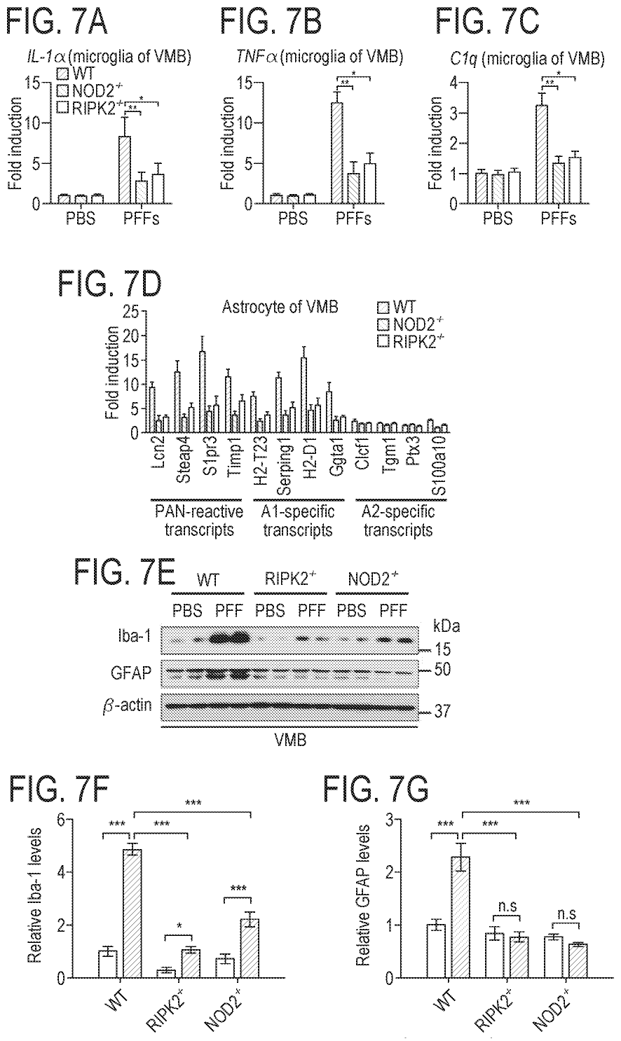

Depletion of NOD2 or RIPK2 significantly ameliorated Lewy body (LB) pathology (FIG. 7A) and suppresses microglia activation (FIG. 7B) in the ventral midbrain of α-synuclein PFFs-induced PD mouse model as assessed by IHC. These results clearly indicate that inhibition of NOD2 and/or RIPK2 activity can be a viable therapeutic target for PD.

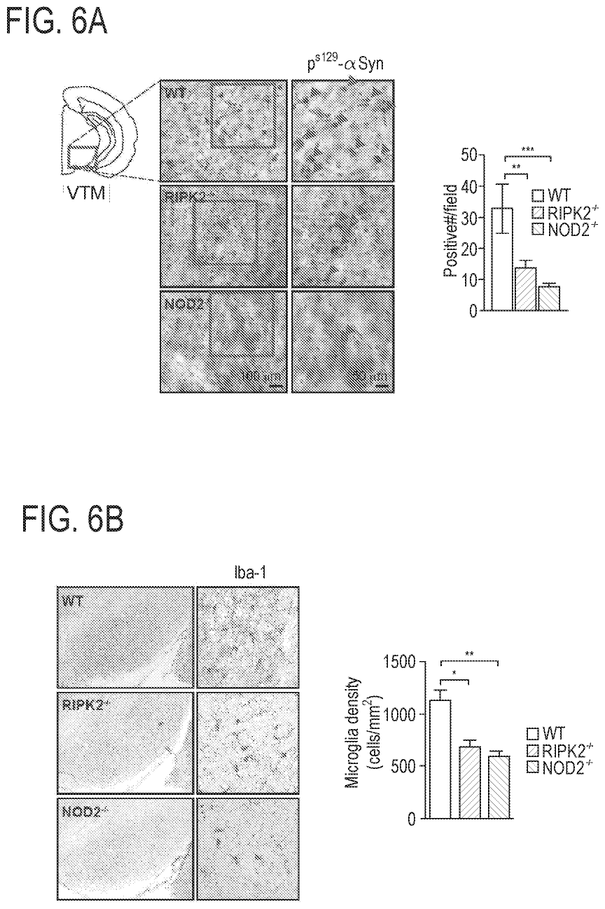

TABLE 20
The positive signals of p-αSyn and microglia density in the
SN (related to FIG. 6A). The values are the mean ± SEM,
n = 5. (*P < 0.05, **P < 0.01, ***P < 0.001).

| | WT + PFFs | RIPK2^{-/-} + PFFs | NOD2^{-/-} + PFFs |
|---|---|---|---|
| # of p-αSyn⁺ | 32.67 ± 13.58 | 13.67 ± 4.163** | 7.67 ± 2.08*** |
| signal | | | |
| # of microglia | 1138.72 ± 91.48 | 683.24 ± 71.52* | 561.82 ± 52.73** |

### Example 7: Depletion of NOD2 or RIPK2 Significantly Suppress Microglia Activation and Reactive Astrocyte Formation in PD Mice

Study rationale and objectives: The aim of this study was to 1) assess the depletion effect of NOD2 or RIPK2 on cytokine production such as TNFα, IL-1α and complement C1q (A1 inducers) in α-synuclein PFFs induced PD mouse model, 2) investigate the depletion effect of NOD2 or RIPK2 on the differentiation of A1 neurotoxic astrocytes in α-synuclein PFFs induced PD mouse model, and 3) investigate the depletion effect of NOD2 or RIPK2 on gliosis in α-synuclein PFFs induced PD mouse model. To explore this, qPCR assay and Western blot analysis were employed.

### Methods

Tissue Lysate Preparation:

Total lysates were prepared by homogenization of tissue in RIPA buffer [50 mM Tris, pH 8.0, 150 mM NaCl, 1% Nonidet P-40, 1% SDS, 0.5% sodium-deoxycholate, phosphatase inhibitor cocktail II and III (Sigma-Aldrich), and complete protease inhibitor mixture (Sigma-Aldrich)]. After homogenization, samples were rotated at 4° C. for 30 min for complete lysis, the homogenate was centrifuged at 22,000×g for 20 min and the supernatants were collected. Protein levels were quantified using the BCA Kit (Pierce, Rockford, Ill., USA) with BSA standards and analyzed by immunoblot.

Comparative Quantitative Real Time PCR (qPCR):

The total RNA from microglia or astrocytes isolated from the ventral mid brain of WT, NOD2 KO, or RIPK2 KO mice with or without α-synuclein PFF injection was extracted with RNA isolation kit (Qiagen, CA) following the instruction provided by the company. RNA concentration was measured spectrophotometrically using NanoDrop 2000 (Biotek, Winooski, Vt.). 1-2 μg of the total RNA were reverse-transcribed to cDNA using the High-Capacity cDNA Reverse Transcription System (Life Technologies, Grand Island, N.Y.). Comparative qPCR was performed in duplicate or triplicate for each sample using fast SYBR Green Master Mix (Life Technologies) and ViiA 7 Real-Time PCR System (Applied Biosystems, Foster City, Calif.). The expression levels of targeted genes were normalized to the expression of β-actin and calculated based on the comparative cycle threshold Ct method (2-ΔΔCt).

Immunoblot Analysis:

Electrophoresis on 8-16% and 4-20% gradient SDS-PAGE gels was performed in order to resolve the obtained 10-20 μg of proteins from the mouse brain tissue. The proteins were then transferred to nitrocellulose membranes. The membranes were blocked with blocking solution (Tris-buffered saline with 5% non-fat dry milk and 0.1% Tween-20) for 1 hr and incubated at 4° C. overnight with anti-Iba-1 (Abcam) and anti-GFAP (EMD Millipore) antibodies, followed by HRP-conjugated rabbit of mouse secondary antibodies (1: 50,000, GE Healthcare, Pittsburgh, Pa., USA) for 1 hr at RT. The bands were visualized by enhanced chemiluminescence (Thermo Scientific, IL, USA). Finally, the membranes were re-probed with HRP-conjugated β-actin antibody (1:40,000, Sigma-Aldrich) after it was stripped.

Results:

Consistent with the in vitro primary microglia results, intrastriatal injection of α-synuclein PFF induces mRNA expression of TNFα, IL-1α and complement C1q, known as reactive A1 astrocyte inducers, in the microglia of the ventral midbrain. This induction is significantly blocked by the depletion of NOD2 or RIPK2 (FIGS. 7A, 7B, and 7C). General astrocyte reactive, A1- and A2-specific mRNA levels were also assessed by qPCR in the primary astrocytes isolated from the ventral midbrain. Intrastriatal injection of α-synuclein PFFs primarily induced A1-specific transcripts and this is prevented by the depletion of NOD2 or RIPK2 (FIG. 7D). Intrastriatal injection of α-synuclein PFFs induces Iba-1, activated-microglia marker, and GFAP, activated astrocytes marker, expression in the ventral midbrain, which is blocked by the depletion of NOD2 or RIPK2 (FIGS. 7E, 7F, and 7G) as assessed by Western blot analysis. These results demonstrate that inhibition of NOD2 and/or RIPK2 suppress activation of both microglia and astrocytes, thus protects neurons in brain.

TABLE 21
mRNA levels (relative fold) of IL-1α (related to FIG. 7A).
The values are the mean ± SEM, n = 4.

| mRNA | Control | PFFs |
|---|---|---|
| WT | 1.00 ± 0.03 | 8.32 ± 2.38 |
| NOD2^{-/-} | 0.92 ± 0.13 | 2.80 ± 1.12 |
| RIPK2^{-/-} | 0.96 ± 0.22 | 3.61 ± 1.42 |

TABLE 22
mRNA levels (relative fold) of TNFα (related to FIG. 7B).
The values are the mean ± SEM, n = 4.

| mRNA | Control | PFFs |
|---|---|---|
| WT | 1.00 ± 0.21 | 12.48 ± 1.36 |
| NOD2^{-/-} | 0.89 ± 0.16 | 3.69 ± 1.48 |
| RIPK2^{-/-} | 1.03 ± 0.17 | 4.92 ± 1.31 |

TABLE 23
mRNA levels (relative fold) of C1q (related to FIG. 7C).
The values are the mean ± SEM, n = 4.

| mRNA | Control | PFFs |
|---|---|---|
| WT | 1.00 ± 0.14 | 3.25 ± 0.41 |
| NOD2^{-/-} | 0.96 ± 0.15 | 1.34 ± 0.23 |
| RIPK2^{-/-} | 1.05 ± 0.12 | 1.52 ± 0.21 |

TABLE 24
The protein expression in the ventral midbrain.
n = 4. (*P < 0.05, **P < 0.01, ***P < 0.001).

| | WT | WT | RIPK2 ^{-/-} | RIPK2 ^{-/-} | NOD2 ^{-/-} | NOD2 ^{-/-} |
|---|---|---|---|---|---|---|
| Proteins | PBS | PFFs | PBS | PFFs | PBS | PFF |
| Iba-1 | 1 ± | 4.86 ± | 0.30 ± | 1.06 ± | 0.72 ± | 2.22 ± |
| | 0.19 | 0.21*** | 0.10 | 0.12*** | 0.17 | 0.28*** |
| GFAP | 1 ± | 2.27 ± | 0.84 ± | 0.77 ± | 0.77 ± | 0.64 ± |
| | 0.10 | 0.26*** | 0.12 | 0.09*** | 0.06 | 0.04*** |

### Example 8: Depletion of NOD2 or RIPK2 Rescues α-Synuclein PFF-Induced Dopaminergic Neurodegeneration and Dopaminergic Terminal Loss In Vivo

Study Rationale and Objectives:

The purpose of this study was to investigate the anti-PD efficacy of NOD2 or RIPK2 depletion in the α-synuclein PFFs induced PD mouse model. To this end, α-synuclein PFFs were injected into the striatum of NOD2 KO or RIPK2 KO mice. Animals at 6 months after α-syn PFF injections were utilized for a variety of neuropathological and neurobehavioral assessments.

### Methods

Mouse Strain for Stereotaxic α-Synuclein PFFs Injection:

NOD2 KO or RIPK2 KO mice was obtained from the Jackson Laboratories (Bar Harbor, Me.). All housing, breeding, and procedures were performed according to the NIH Guide for the Care and Use of Experimental Animals and approved by the Johns Hopkins University Animal Care and Use Committee.

α-Synuclein Protein Purification and PFF Preparation:

Recombinant mouse α-synuclein proteins were purified as previously described with an IPTG-independent inducible pRK172 vector system. Endotoxin was depleted by ToxinEraser endotoxin removal kit (Genscript, NJ, USA). α-synuclein PFFs (5 mg ml⁻¹) was prepared in PBS while stirring with a magnetic stirrer (1,000 rpm at 37° C.). After a week of incubation of the α-synuclein protein, aggregates were diluted to 0.1 mg ml⁻¹ with PBS and sonicated for 30 s (0.5 s pulse on/off) at 10% amplitude (Branson Digital sonifier, Danbury, Conn., USA). α-synuclein PFFs was validated using atomic force microscopy and transmission electron microscopy, and the ability to induce phospho-serine 129 α-synuclein (p-α-synSer129) was confirmed using immunostaining. α-synuclein PFFs was stored at −80° C. until use.

Stereotaxic α-Synuclein PFFs Injection:

For stereotaxic injection of α-synuclein PFFs, 3 months old NOD2 or RIPK2 KO male and female mice were anesthetized with xylazene and ketamine. An injection cannula (26.5 gauge) was applied stereotaxically into the striatum (STR) (mediolateral, 2.0 mm from bregma; anteroposterior, 0.2 mm; dorsoventral, 2.6 mm) unilaterally into the right hemisphere. The infusion of 2 μL α-synuclein PFFs (2.5 μg/mL in PBS) or the same volume of PBS was performed at a rate of 0.2 μL per min. After the final dose, the injection cannula was maintained in the STR for additional 5 min for a complete absorption of the α-synuclein PFFs or PBS then slowly removed from the mouse brain. The head skin was closed by suturing and wound healing and recovery were monitored following surgery. For stereological analysis, animals were perfused and fixed intracardially with ice-cold PBS followed by 4% paraformaldehyde at 6 months after striatal α-synuclein PFFs injections. The brain was removed and processed for immunohistochemistry or immunofluorescence. Behavioral tests were performed at 6 months after the unilateral striatal α-synuclein PFFs injections.

Tyrosine Hydroxylase (TH) Immunohistochemistry and Quantitative Analysis:

Mice were perfused with ice-cold PBS followed by fixed with 4% paraformaldehyde/PBS (pH 7.4). Brains were collected and post-fixed for overnight in the 4% paraformaldehyde and incubated in 30% sucrose/PBS (pH 7.4) solution. Brains were frozen in OCT buffer and 30 μm serial coronal sections were cut with a microtome. Free-floating 30 μm sections were blocked with 4% goat or horse serum/PBS plus 0.2% Triton X-100 and incubated with an antibody against TH (Novus Biologicals, Littleton, Colo., USA) followed by incubation with biotin-conjugated anti-rabbit antibody (Vectastain Elite ABC Kit, Vector laboratories, Burlingame, Calif., USA). After developed using SigmaFast DAB Peroxidase Substrate (Sigma-Aldrich), sections were counterstained with Nissl (0.09% thionin). TH-positive and Nissl positive DA neurons from the SNc region were counted through optical fractionators, the unbiased method for cell counting by using a computer-assisted image analysis system consisting of an Axiophot photomicroscope (Carl Zeiss Vision) equipped with a computer controlled motorized stage (Ludl Electronics), a Hitachi HV C20 camera, and Stereo Investigator software (MicroBright-Field). Fiber density in the striatum was analyzed by optical density (OD) measurement using ImageJ software (NIH, http://rsb.info.nih.gov/ij/).

Immunoblot Analysis:

The mouse brain tissues were homogenized and prepared in lysis buffer [(10 mM Tris-HCL, pH 7.4, 150 mM NaCl, 5 mM EDTA, 0.5% Nonidet P-40, 10 mM Na-β-glycerophosphate, phosphate inhibitor mixture I and II (Sigma-Aldrich, St. Louis, Mo., USA), and complete protease inhibitor mixture (Roche), using a Diax 900 homogenizer (Sigma-Aldrich, St. Louis, Mo., USA). After homogenization, samples were rotated at 4° C. for 30 min for complete lysis, the homogenate was centrifuged at 15,000 rpm for 20 min and the supernatants were collected. Protein levels were quantified using the BCA Kit (Pierce, Rockford, Ill., USA) with BSA standards and analyzed by immunobloting. Electrophoresis on 8-16% and 4-20% gradient SDS-PAGE gels were performed in order to resolve the obtained 10-20 μg of proteins from the mouse brain tissue. The proteins were transferred to nitrocellulose membranes. The membranes were blocked with blocking solution (Tris-buffered saline with 5% non-fat dry milk and 0.1% Tween-20) for 1 h and incubated at 4° C. overnight with anti-TH (1:2000, Novus Biologicals, Littleton, Colo., USA), anti-DAT, followed by HRP-conjugated rabbit of mouse secondary antibodies (1: 50000, GE Healthcare) and HRP-conjugated mouse of donkey secondary antibodies (1: 10000, GE Healthcare) for 1 h at room temperature. The bands were visualized by enhanced chemiluminescence (Thermo Scientific). Finally, the membranes were re-probed with HRP-conjugated β-actin antibody (1:50,000, Sigma-Aldrich, St. Louis, Mo., USA) after it was stripped.

Pole Test:

Mice were acclimatized in the behavioral procedure room for 30 min. The pole was made with 75 cm of metal rod at diameter of 9 mm. Mice were placed on the top of the pole (7.5 cm from the top of the pole) facing the head-up. Total time taken to reach the base of the pole was recorded. Before actual test, mice were trained for two consecutive days. Each training session consisted of three test trials. On the test day, mice were evaluated in three sessions and total time was recorded. The maximum cutoff time to stop the test and recording was 60 sec. Results for turn down, climb down, and total time (in sec) were recorded.

Grip Strength Test:

Neuromuscular strength test was performed using a Bioseb grip strength test machine (BIO-G53, Bioseb, FL USA). Performance of mice was assessed three times. To assess grip strength, mice were allowed to grasp a metal grid with either by their fore limbs or both fore and hind limbs. The tail was gently pulled and the maximum holding force recorded by the force transducer when the mice released their grasp on the grid. The peak holding strength was digitally recorded and displayed as force in grams Grip strength was scored as grams (g) unit.

Results:

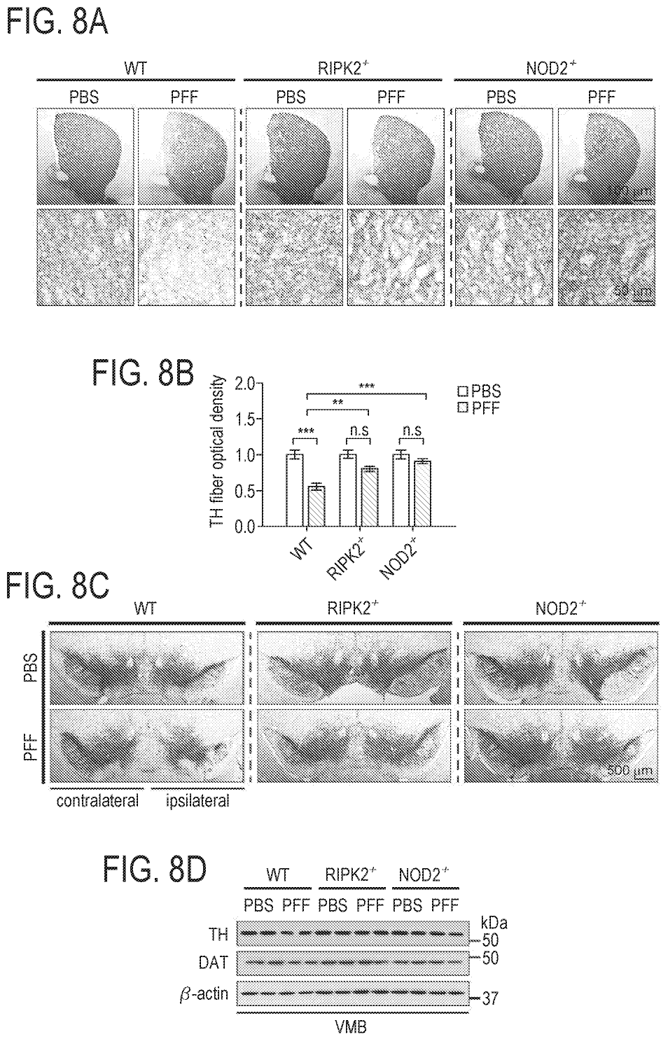

α-synuclein PFTs-induced reduction in striatal tyrosine hydroxylase immunoreactivity are rescued by depletion of NOD2 or RIPK2 (FIGS. 8A and 8B). Western blot analysis reveals that the α-synuclein PFFs-mediated reduction in tyrosine hydroxylase and dopamine transporter (DAT) immunoreactivity is restored by depletion of NOD2 or RIPK2 in the ventral midbrain (FIG. 8D). α-synuclein PFFs injection induces a significant loss of tyrosine hydroxylase- and Nissl-positive neurons in the SNpc, which is prevented by depletion of NOD2 or RIPK2 (FIGS. 8C, 8E, and 8F). Depletion of NOD2 or RIPK2 also significantly reduces the behavioral deficits elicited by α-synuclein PFF injection as measured by the grip strength (FIG. 8G) and the pole test (FIG. 8H), These results clearly indicate that inhibition of NOD2 and/or RIPK2 activity protects neurons and ameliorates PD in vivo.

TABLE 25
The optical density of TH positive fiber, the number of dopaminergic neurons,
protein expression, and behavioral deficits (related FIGS. 8B, 8E, 8F, 8G, 8H,
8I, and 8J). n = 5. (*P < 0.05, **P < 0.01, ***P < 0.001).

| | WT | WT | RIPK2 ^{-/-} | RIPK2 ^{-/-} | NOD2 ^{-/-} | NOD2 ^{-/-} |
|---|---|---|---|---|---|---|
| | PBS | PFFs | PBS | PFFs | PBS | PFF |
| TH fiber | 1 ± 0.06 | 0.55 ± | 1 ± 0.05 | 0.80 ± | 1 ± 0.06 | 0.91 ± |
| optical density | | 0.05*** | | 0.03** | | 0.03*** |
| Neuron | | | | | | |
| cells count | | | | | | |
| TH⁺ | 6563 ± | 3775 ± | 6806 ± | 5844 ± | 7075 ± | 6613 ± |
| | 301.5 | 325.0*** | 168.1 | 126.9*** | 261.6 | 318.8*** |
| Nissl⁺ | 8531 ± | 5338 ± | 8881 ± | 7381 ± | 8650 ± | 8269 ± |
| | 634.7 | 623.9*** | 458.0 | 316.1*** | 690.2 | 539.6*** |
| Proteins | | | | | | |
| TH | 1 ± 0.05 | 0.59 ± | 1 ± 0.10 | 0.90 ± | 1 ± 0.07 | 0.91 ± |
| | | 0.07* | | 0.15* | | 0.03*** |
| DAT | 1 ± 0.08 | 0.61 ± | 1 ± 0.03 | 0.85 ± | 1 ± 0.05 | 0.96 ± |
| | | 0.03* | | 0.07** | | 0.05** |
| Behavior tests | | | | | | |
| Pole test | 9.07 ± | 20.15 ± | 10.12 ± | 12.53 ± | 10.24 ± | 10.58 ± |
| | 0.78 | 2.18*** | 1.01 | 2.57** | 1.57 | 1.61*** |
| Grip strength | 145.4 ± | 112.0 ± | 141.4 ± | 130.0 ± | 138.5 ± | 138.4 ± |
| test | 3.99 | 3.81*** | 3.19 | 5.57** | 3.88 | 4.41*** |

### Example 9: Orally Administered RIPK2 Inhibitor Ameliorates LB Pathology and Suppresses Microglia Activation in α-Synuclein PFFs-Induced PD Animal Model

Study rationale and objectives: The purpose of this study was to investigate the anti-PD efficacy of Gefitinib, RIPK2 inhibitor, in the α-synuclein PFF model PD. To this end, α-synuclein PFF were injected into the striatum of NOD2 KO or RIPK2 KO. α-synuclein PFFs-induced PD mice were orally treated with Gefitinib (Gef) (30 mg/kg, once daily) after 1 month striatal α-synuclein PFF injection for 5 months and tissues were analyzed.

### Methods

Mouse Strain for Stereotaxic α-Synuclein PFFs Injection:

NOD2 or RIPK2 KO mice was obtained from the Jackson Laboratories (Bar Harbor, Me.). All housing, breeding, and procedures were performed according to the NIH Guide for the Care and Use of Experimental Animals and approved by the Johns Hopkins University Animal Care and Use Committee.

α-Synuclein Protein Purification and PFF Preparation:

Recombinant mouse α-synuclein proteins were purified as previously described with an IPTG-independent inducible pRK172 vector system. Endotoxin was depleted by ToxinEraser endotoxin removal kit (Genscript, NJ, USA). α-synuclein PFFs (5 mg ml⁻¹) was prepared in PBS while stirring with a magnetic stirrer (1,000 rpm at 37° C.). After a week of incubation of the α-synuclein protein, aggregates were diluted to 0.1 mg ml⁻¹ with PBS and sonicated for 30 s (0.5 s pulse on/off) at 10% amplitude (Branson Digital sonifier, Danbury, Conn., USA). α-synuclein PFFs was validated using atomic force microscopy and transmission electron microscopy, and the ability to induce phospho-serine 129 α-synuclein (p-α-synSer129) was confirmed using immunostaining. α-synuclein PFFs was stored at −80° C. until use.

Stereotaxic α-synuclein PFFs injection and Immunohistochemistry (IHC): For stereotaxic injection of α-synuclein PFFs, 3 months old NOD2 KO or RIPK2 KO male and female mice were anesthetized with xylazene and ketamine. An injection cannula (26.5 gauge) was applied stereotaxically into the striatum (STR) (mediolateral, 2.0 mm from bregma; anteroposterior, 0.2 mm; dorsoventral, 2.6 mm) unilaterally into the right hemisphere. The infusion of 2 μL α-synuclein PFFs (2.5 μg/mL in PBS) or the same volume of PBS was performed at a rate of 0.2 μL per min. After the final dose, the injection cannula was maintained in the STR for additional 5 min for a complete absorption of the α-synuclein PFFs or PBS then slowly removed from the mouse brain. The head skin was closed by suturing and wound healing and recovery were monitored following surgery. For IHC analysis, animals were perfused and fixed intracardially with ice-cold PBS followed by 4% paraformaldehyde at 3 months after striatal α-synuclein PFFs injections. The brain was removed and processed for immunohistochemistry. IHC for pS129-α-synuclein immunoreactivity was performed at 3 months after the unilateral striatal α-synuclein PFFs injections. Treatment of Gefitinib was accomplished after one month of unilateral striatal α-synuclein PFFs injection, once daily.

Results:

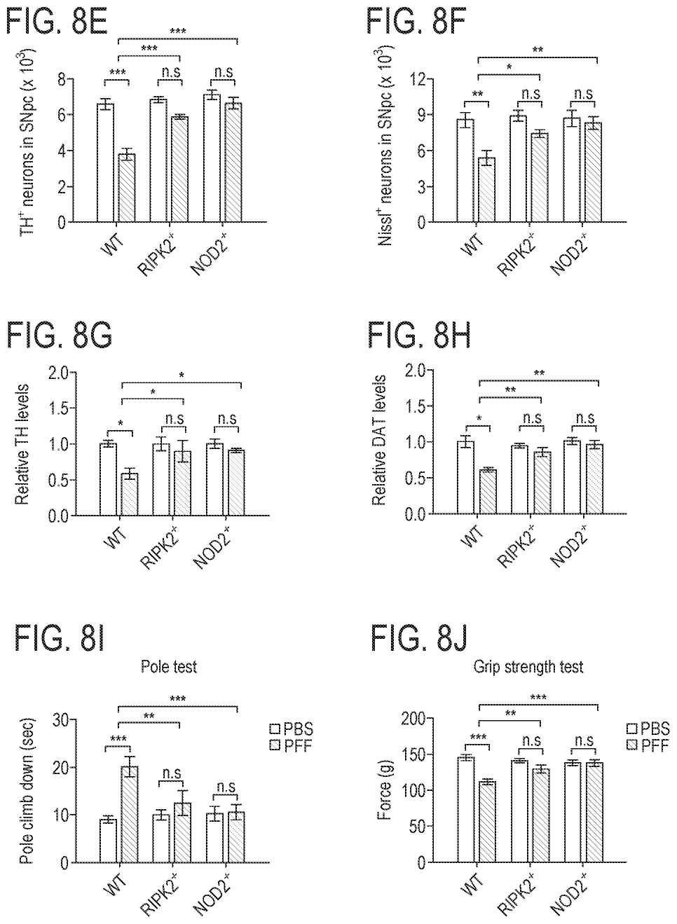

Gefitinib treatment significantly ameliorates LB pathology (FIG. 9A) as evidenced by reduced pS129-α-synuclein immunoreactivity and suppresses microglia activation (FIG. 9B) in the ventral midbrain compared to that of non-treated PD mice as assessed by IHC. These results demonstrate that RIPK2 inhibitors are potential drugs for neurodegenerative disorders associated with microglia activation such as PD.

TABLE 26
The positive signals of p-αSyn and microglia in the
SN (related to FIG. 9A). The values are the
mean ± SEM, n = 5. (*P < 0. 05)

| | Veh + PFFs | Gefitinib + PFFs |
|---|---|---|
| # of p-αSyn | 32.14 ± 1.93 | 16.49 ± 2.01* |
| positive signal | | |
| # of microglia | 1138.72 ± 91.48 | 409.15 ± 94.27* |

### Example 10: p-RIPK2 is Elevated in the Hippocampus of Human AD Postmortem Tissues

Study Rationale and Objectives:

The aim of this study was to investigate expressions of phosphorylated RIPK2 (p-RIPK2) in post-mortem human brain tissues of patients with AD. To explore this, IHC was employed.

### METHODS

IHC for AD Postmortem Brain:

Slides with 10-μm thickness of formalin-fixed paraffin-embedded human postmortem hippocampus tissues (n=3 for each of control and AD) were obtained from the Division of Neuropathology, Department of Pathology, Johns Hopkins University. The tissue sections were deparaffinized and rehydrated, and then heat-induced epitope retrieval was performed with citrate-based antigen unmasking solutions (Vector Laboratories). Then, the slides were stained with rabbit polyclonal pRIPK2 antibody. All sections were stained with hematoxylin.

Results:

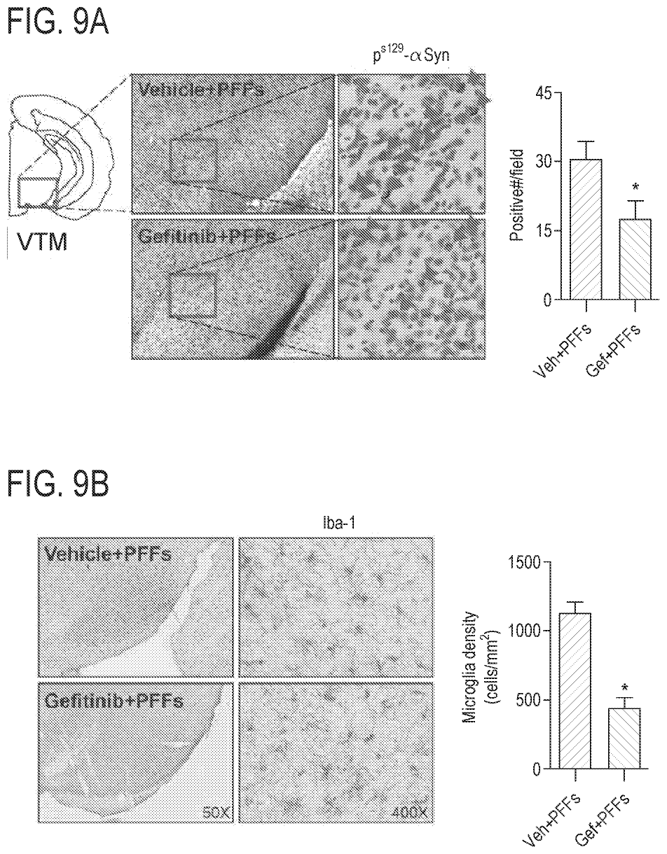

Our data indicates that p-RIPK2 immunoreactivity are significantly increased in the hippocampus from AD patient samples as assessed by IHC (FIGS. 10A, B), suggesting that excessive RIPK2 activation plays a pivotal role in the pathogenesis of AD. These results indicate that targeting RIPK2 and/or p-RIPK2 activity can be a viable therapeutic target for neurodegenerative disorders, including AD.

TABLE 27
The intensity of p-RIPK2 in the hippocampus
of AD postmortem (related to FIG. 10A).
The values are the mean ± SEM, n = 9. (***P < 0.001).

| Relative intensity | Control | AD |
|---|---|---|
| p-RIPK2 | 1.00 ± 0.08 | 5.76 ± 0.46*** |

### Example 11: Amyloid-β (Aβ or Abeta) Aggregates-Activated Microglia Induce mRNA RIPK2 and Inflammatory Cytokines

Study rationale and objectives: The aim of this study was to confirm that microglia activated by Abeta aggregates induce mRNA RIP2K along with inflammatory cytokines.

### Methods

Synthetic Abeta₁₋₄₂ oligomers were generated as previously described (PMID:27834631). Hydroxyfluroisopropanol (HFIP)-treated synthethic Abeta₁₋₄₂ peptides (rPeptide, Bogart, Ga., USA) were dissolved in dimethylsulfoxide (DMSO) and further diluted in phosphate-buffered saline (PBS) to obtain a 250 μM stock solution. The stock solution was incubated at 4° C. for at least 24 hours and stored at −80° C. until use. Before use, the solution was centrifuged at 12,000 g for 10 minutes and the supernatant was used as an oligomeric Aβ.

BV-2 microglial cells were cultured in DMEM media. 10⁶ of BV-2 microglia in 6 well plate were treated with 2.5 μM of Abeta for 4 hrs. Total RNA from cultured cells was extracted with a RNA isolation kit (Qiagen, Valencia, Calif., USA) following manufacturer's instructions. RNA concentration was measured spectrophotometrically using a NanoDrop 2000 (Thermo scientific). Subsequently, 2 μg of total RNA was reverse transcribed to cDNA using the High-Capacity cDNA Reverse Transcription System (Life Technologies, Grand Island, N.Y., USA). Comparative qPCR was performed using fast SYBR Green Master Mix (Life Technologies) and steponeplus real-time per system (Applied Biosystems, Foster City, Calif., USA). The expression levels of target genes were normalized to the expression of GAPDH and calculated based on the comparative cycle threshold Ct method^{-ΔΔ(2)}Ct. (n=3)

Results:

To determine the potential mechanism of action of RIPK2 in microglia, the expression of RIPK2 was assessed in BV-2 microglia cells. The mRNA expression of RIPK2 was significantly increased when BV-2 microglia were activated by Aβoligomer (AβO). AβO increases RIPK2 mRNA expression almost 10-fold in microglia. Along with the expression of RIPK2, multiple inflammatory mediators were measured. AβO increased the level of a subset of cytokines including TNF-α, IL-1β and IL-6, typical markers of M1 microglia.

TABLE 28
Abeta-activated microglia induces RIPK2 and
inflammatory cytokines.

| mRNA | PBS | AβO |
|---|---|---|
| RIPK2 | 1 | 9.5 ± 2.6 |
| TNFα | 1 | 19.6 ± 6.2 |
| IL-1α | 1 | 41.48 ± 16.8 |
| IL-6 | 1 | 22.6 ± 7.3 |

### Example 12: Amyloid-β (Aβ) Aggregates-Activated Microglia Induce Phosphorylation of RIPK2

Study Rationale and Objectives:

The aim of this was to confirm that microglia activated by Abeta aggregates induce phosphorylated RIPK2 (p-RIPK2) and NOD2.

### Methods

2×10⁶ of BV-2 microglia in 6 well plate were treated with 5 μM of Aβ for 15, 60, 120, 240 or 360 min. Subsequently, cell lysates were lysed by RIPA buffer with complete, Mini, EDTA-free Protease Inhibitor Cocktail (Sigma) for 30 min, incubated with anti-RIPK2 antibody overnight followed by Protein A/G incubation for 3 hrs and analyzed with western blotting with anti-phospho-specific RIP2K or NOD2 antibody.

Results:

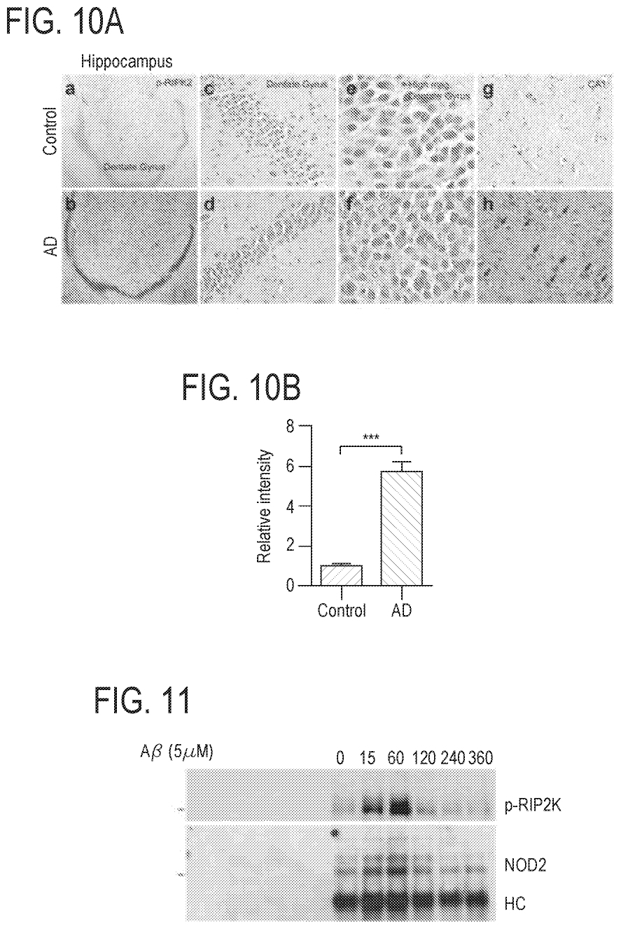

As seen in FIG. 11, p-RIPK2 appeared from 15 min after Aβ treatment with peaked at 60 min. Consistent with the RIPK2 phosphorylation, binding of NOD2 increased along with the phosphorylation when microglial cells were treated with AβO. This result indicates the chain reaction of NOD2 binding to RIPK2 followed by phosphorylation for AβO-induced activation in microglia cells in AD.

### Example 13. Depletion of NOD2 or RIPK2 Suppress AβO-Induced Microglia Activation

Study Rationale and Objectives:

The aim of this study was to 1) assess the depletion effect of NOD2 or RIPK2 on cytokine production such as TNFα and IL-6 (A1 inducers) in microglia activated by AβO. To explore this, qPCR assay was employed.

### Methods

In this study, Wild-type (WT), NOD2 knockout (B6.129S1-Nod2tm1Flv/J, NOD2^{-/-}), and RIPK2 knockout (B6.12951-Nod2tm1Flv/J, RIPK2^{-/-}) mice were accessed from The Jackson Laboratory. For primary microglial culture, whole brains from mouse pups at postnatal day 2 (P2) were obtained. After removal of the meninges, the brains were washed in DMEM (Cellgro) supplemented with 10% heat-inactivated FBS, 50 U ml⁻¹ penicillin, 50 μg ml⁻¹ streptomycin. The brains were transferred to 0.25% trypsin-EDTA and incubated for 10 min. DMEM complete medium was used to neutralize Trypsin. A single-cell suspension was obtained by pipetting. Cell debris and aggregates were removed by passing the single-cell suspension through a 70-μm nylon mesh. The final single-cell suspension thus achieved was cultured in T175 flasks for 2 weeks, with a complete medium change on day 7. The mixed glial cell population was separated into astrocyte-rich and microglia-rich fractions using the EasySep Mouse CD11b Positive Selection Kit (StemCell). The magnetically separated fractions of microglia were culture. Primary cultured microglia from wild-type (WT), NOD2 knockout (NOD2^{-/-}), and RIPK2 knockout (RIPK2^{-/-}) mice were activated with 5 μM of AβO for 4 hours. The gene expression of TNFα and IL-6 was measured by real-time RT-PCR. The values are the mean±SD of four independent experiments.

Results:

To validate the target signaling of NOD2-RIP2K pathway in AD, primary microglia were activated by AβO followed by real-time PCR for TNFα and IL-6 was accessed. AP 42 oligomer (AβO), activated microglia upregulated the mRNA levels of TNFα and IL-6 in microglia from WT littermate. Depletion of NOD2 or RIPK2 significantly reduced levels of pro-inflammatory cytokines in primary microglia activated with AβO. This result indicates that inhibition of NOD2-RIPK2 signaling shuts down the release of proinflammatory and toxic mediators induce the AβO-induced toxicity.

TABLE 29
mRNA levels (relative fold) of TNF-a and IL-6 in normal (PBS) and Aβ activated
mouse primary microglia of WT, RIP2, or NOD2 Knockout mice. The values are
the mean ± SD, n = 2-4. (*P < 0.05 vs. PBS).

| | WT | | RIP2K KO | | NOD2 KO | |
|---|---|---|---|---|---|---|
| | PBS | AβO | PBS | AβO | PBS | AβO |
| TNF-α | 1 ± 0.18 | 1.82 ± 0.48* | 1 ± 0.11 | 0.8 ± 0.13 | 1 ± 0.30 | 1.08 ± 0.19 |
| IL-6 | 1 ± 0.09 | 1.76 ± 0.16* | 1 ± 0.018 | 1.15 ± 0.50 | 1 ± 0.33 | 0.28 ± 0.11* |

### Example 14: Inhibitors of RIPK2 Suppress APO-Induced Microglia Activation

Study rationale and objectives: The object of this study was to 1) assess the effect of RIPK2 inhibitors on cytokine production such as TNFα, IL-6 and complement C1q (reactive A1 astrocyte inducers) by primary microglia activated with Aβ aggregates. To this end, qPCR assays were employed.

### Methods

To examine the effect of RIPK2 inhibition, 10⁶ of BV-2 microglia in 6 well plate were preincubated with DMSO, GSK583(1 μM, Medchemexpress), OD361 (1 Calbiochem), or Sorafenib (1 μM) for one hour. For mRNA analysis, 5 μM of AβO was treated additional for 4 hours.

Results: To confirm the anti-inflammatory efficacy of RIPK2 inhibition in BV-2 microglia activated by abnormally aggregated proteins, e.g. AβO, real-time PCR for TNFα, IL-6, and C1q was accessed. Aβ 42 oligomer (AβO), activated microglia upregulated the mRNA levels of C1q, IL-6 and TNF-α. Importantly, when microglia are pretreated with RIPK2 inhibitors, GSK583 (1 μM), OD36 (1 μM) or Sorafenib (1 μM) followed by AβO (5 μM) blocked microglial activation and significantly reduced the release of multiple inflammatory mediators including C1q, IL-6 and TNFα. Consistent with the study results in ELISA, RIPK2 inhibitor treated AβO activated microglia demonstrated significantly reduced the expression of pro-inflammatory markers as summarized in Table 30. This result indicates that inhibition of RIPK2 activity by RIPK2 inhibitors block microglia activation that can induce reactive A1 reactive astrocyte formation and neuronal damage in neurodegenerative disorders including PD and AD.

TABLE 30
Effects of RIPK2 inhibitor in Aβ activated microglia. mRNA
levels of C1q, IL-6 and TNF-α in BV-2 microglia were
analyzed by real-time PCR. ±SD, n = 2-4 per groups. (Ctrl
vs, **P < 0.01, ***P < 0.001, Aβ vs, ^{#}P < 0.05, ^{##}P < 0.01,
^{###}P < 0.001).

| | PBS | Abeta | | | |
|---|---|---|---|---|---|
| | PBS | PBS | GSK583 | OD36 | Sorafenib |
| C1q | 1 ± 0.13 | 3.7 ± 0.06 | 0.87 ± 0.14 | 0.85 ± 0.12 | 3.3 ± 0.09 |
| IL-6 | 1 ± 0.08 | 12.53 ± 0.59 | 4.89 ± 0.23 | 7.64 ± 0.48 | 7.13 ± 0.71 |
| TNFα | 1 ± 0.16 | 16.19 ± 3.21 | 3.23 ± 0.27 | 5.85 ± 0.03 | 9.69 ± 0.47 |

### Example 15: RIPK2 is Elevated in the Brain of 5×-FAD AD Transgenic Mice

Study Rationale and Objectives:

The purpose of this study was to confirm elevated RIPK2 in transgenic AD mouse model as shown in PD mouse models.

### Methods

Animals: 5×FAD (Tg6799, B6SJL-Tg(APPSwF1Lon, PSEN1*M146L*L286V) 6799Vas/Mmjax) mice were obtained from Jackson Lab. These widely used mice contain five mutations, overexpress mutant human APP(695) with the Swedish (K670N, M671L), Florida (I716V), and London (V717I) Familial AD mutations along with human PS1 harboring two FAD mutations, M146L and L286V. 5XFAD mice recapitulate major features of AD amyloid pathology and is known as a useful model of intraneuronal Abeta-42 induced neurodegeneration and amyloid plaque formation. Aβ deposition is progressive and appear intracellularly as early as three of four months of age and extracellular deposits appear by six months in the frontal cortex and become more extensive by twelve months. In this study, 6-month old male 5×FAD AD mice were used.

Expression of RIP-kinase: Total RNA was isolated from hippocampus of 6 months age wild-type or 5×FAD mice and differential gene expressions including RIPK1, RIPK2, RIPK3 and NOD2 were assessed using real-time PCR. The levels of mRNA were normalized to the housekeeping gene 18S rRNA. The protein expression levels of RIP-kinases were access with Western blotting from the cortex region of seven months age wild-type (WT) or 5×FAD mice.

Results: mRNA expression of RIPK1, RIPK2, RIPK3 and NOD2 in 5×FAD mice was compared with the WT littermate mice. RIPK1 and RIPK2 significantly increased in 5×FAD compared to that of WT littermate, indicating that the RIP kinases are a viable therapeutic target for neurodegenerative diseases including AD and PD. To assess the change of RIPK protein expressions, cortex region of seven months 5×FAD was analyzed. Protein expression of RIPK2 significantly increased in 5×FAD compared to that of RIPK1 or RIPK2.

### Example 16: Depletion of NOD2 or RIPK2 Rescues Cognitive Impairments in AβO-Induced AD Mice

Study Rationale and Objectives:

The purpose of this study was to investigate the anti-AD efficacy of NOD2 or RIPK2 depletion in the AβO-induced AD mouse model. To this end, AβO were injected into the striatum of control, NOD2 KO or RIPK2 KO mice. Animals at 2 weeks after AβO injections were utilized for a variety of neurobehavioral assessments.

### Methods

Preparation of Abeta₁₋₄₂ Oligomer:

Synthetic Abeta₁₋₄₂ oligomers (AbetaO₁₋₄₂) were generated as previously described. Hydroxyfluroisopropanol (HFIP)-treated synthetic Abeta₁₋₄₂ peptides (rPeptide, Bogart, Ga., USA) were dissolved in dimethylsulfoxide (DMSO) and further diluted in phosphate-buffered saline (PBS) to obtain a 250 μM stock solution. The stock solution was incubated at 4° C. for at least 24 hours and stored at −80° C. until use. Before use, the solution was centrifuged at 12,000 g for 10 minutes and the supernatant was used as an oligomeric Aβ.

Stereotaxic AbetaO₁₋₄₂ i.c.v. Injection:

For stereotaxic injection of AbetaO₁₋₄₂, 3 months old NOD2 or RIPK2 KO male and female mice were anesthetized with xylazene and ketamine. An injection cannula (26.5 gauge) was applied stereotaxically into the intracerebroventricular (i.c.v.) space, with coordinates 0.2 mm posterior and 1.0 mm lateral from the bregma and 2.5 mm ventral from the skull surface (Paxinos and Franklin, The Mouse Brain in Stereotaxic Coordinates, 2^{nd} Ed., Academic Press, San Diego (2001)). The infusion of 5 μL AbetaO₁₋₄₂ (0.5 μmol) or the same volume of PBS was performed at a rate of 0.2 μL per min. After the final dose, the injection cannula was maintained in the i.c.v for additional 5 min for a complete absorption of the AbetaO₁₋₄₂ or PBS then slowly removed from the mouse brain. The head skin was closed by suturing and wound healing and recovery were monitored following surgery. Behavioral tests were performed at seven days after the bilateral i.c.v. AbetaO₁₋₄₂ injections (total 5 μmol).

Morris Water Maze Test (MWMT):

The MWMT was performed as described in the previous report (Vorhees and Williams, Nat. Protoc. 1:848-58 (2006)). The MWM is a white circular pool (150 cm in diameter and 50 cm in height) with four different inner cues on surface. The circular pool was filled with water and a nontoxic water-soluble white dye (20±1° C.) and the platform was submerged 1 cm below the surface of water so that it was invisible at water level. The pool was divided into four quadrants of equal area. A black platform (9 cm in diameter and 15 cm in height) was centered in one of the four quadrants of the pool. The location of each swimming mouse, from the start position to the platform, was digitized by a video tracking system (ANY-maze, Stoelting Co., Wood Dale, Ill., USA). The day before the experiment was spend to swim training for 60 sec in the absence of the platform. The mice were then given two trial sessions each day for four consecutive days, with an inter-trial interval of 15 min, and the escape latencies were recorded. This parameter was averaged for each session of trials and for each mouse. Once the mouse located the platform, it was permitted to remain on it for 10 sec. If the mouse was unable to locate the platform within 60 sec, it was placed on the platform for 10 sec and then returned to its cage by the experimenter. On day 6, the probe trial test involved removing the platform from the pool and mice were allowed the cut-off time of 60 sec.

Results:

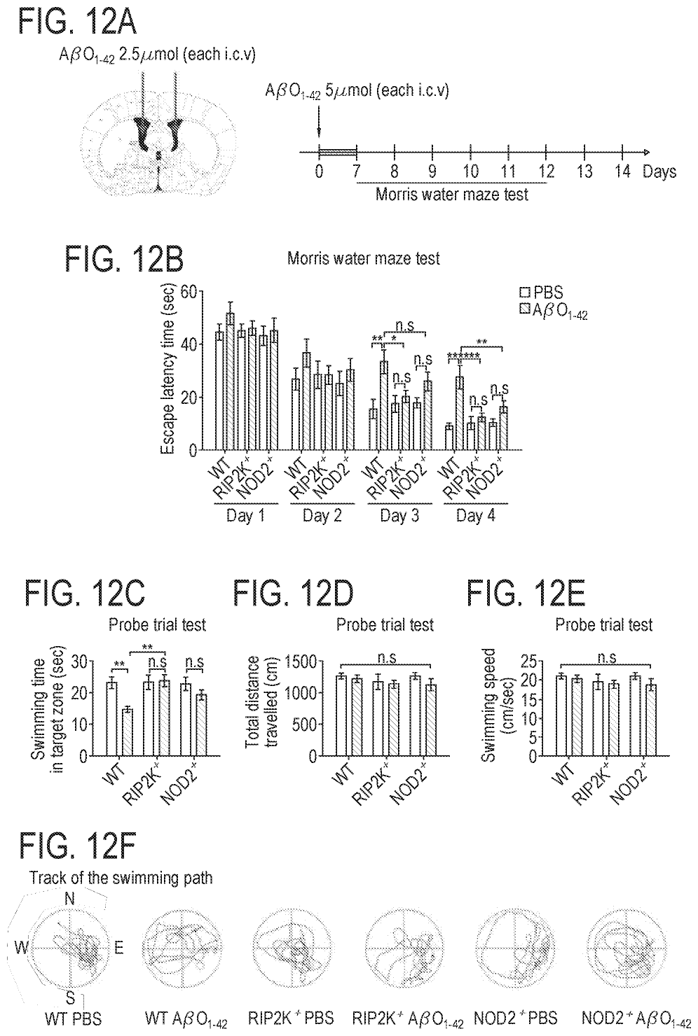

We assessed spatial learning and memory by the Morris Water Maze task seven days after AβO₁₋₄₂ or PBS injection. On the first day of exposure to the Morris Water Maze, there is no difference in finding the platform between AβO₁₋₄₂ or PBS injected wild type, RIPK2^{-/-} or NOD2^{-/-} mice (FIG. 12B). On day 3 and 4 of exposure to the Morris Water Maze the AβO₁₋₄₂ injected wild type mice demonstrated a significantly increased escaped latency time compared the PBS treated wild type mice (FIG. 12B). In contrast, both the AβO₁₋₄₂ injected RIPK2^{-/-} and NOD2^{-/-} mice showed escape latency times comparable to that of PBS wild type mice. Following the last day of trial sessions (Day 5), both AβO₁₋₄₂ injected RIPK2^{-/-} and NOD2^{-/-} mice demonstrated significantly increased swimming time and paths in the target quadrant after the platform was removed, similar to that of PBS injected wild type mice compared to AβO₁₋₄₂ injected wild type mice (FIGS. 12C and 12F). The swimming speed and total distance traveled did not show significant differences among all experimental groups (FIGS. 12D and 12E). These results clearly indicate that inhibition of RIPK2 and/or NOD2 activity improves memory functions and rescues cognitive impairments in AD models.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A method of preventing or treating a neurodegenerative disease or disorder, comprising: administering to a subject in need thereof a therapeutically effective amount of a Receptor-Interacting Protein (RIP) kinase 2 (RIPK2) inhibitor, wherein the neurodegenerative disease or disorder is associated with upregulated NOD2, phosphorylated RIPK2, and/or RIPK2 in one or more regions of the central nervous system (CNS).

2. The method of claim 1, wherein the RIPK2 inhibitor inhibits RIPK2 activity and/or expression.

3. The method of claim 1, wherein the RIPK2 inhibitor is selective over RIP kinase 1 and/or RIP kinase 3.

4. The method of claim 1, wherein the RIPK2 inhibitor is administered in an amount effective in inhibiting one or more activities selected from NOD1-dependent activation of NFκB, NOD2-dependent activation of NF-kB, microglial activation, and/or reactive astrocytes formation.

5. A method for treating a neurodegenerative disease or disorder associated with activation of central nervous system (CNS) resident innate immune cells by abnormally aggregated proteins, the method comprising administering to a subject in need thereof an effective amount of a Receptor-Interacting Protein (RIP) kinase 2 (RIPK2) inhibitor.

6. The method of claim 5, wherein the RIPK2 inhibitor is administered in an amount effective to inhibit the activation of CNS resident innate immune cells by abnormally aggregated proteins.

7. The method of claim 5, wherein the administering of the RIPK2 inhibitor reduces the level of one or more inflammatory or neurotoxic mediators secreted from the activated innate immune cells that induce neuro-inflammation and neuronal damage.

8.-10. (canceled)

11. The method of claim 5, wherein the neurodegenerative disease or disorder is Parkinson's disease or Alzheimer's disease.

12. A method of inhibiting activation of central nervous system (CNS) resident innate immune cells by abnormally aggregated proteins, the method comprising contacting the CNS resident innate immune cells with an effective amount of a Receptor-Interacting Protein (RIP) kinase 2 (RIPK2) inhibitor.

13.-15. (canceled)

16. The method of claim 12, wherein the amount of RIPK2 inhibitor is effective to reduce the level of one or more inflammatory or neurotoxic mediators secreted by the CNS resident innate immune cells compared to a control, wherein the one or more inflammatory or neurotoxic mediators are TNFα, IL-1α, IL-1β, C1q, IL-6, and/or combinations thereof.

17. A method of treating Parkinson's disease in a subject in need thereof, the method comprising administering to the subject a therapeutically effective amount of a RIPK2 inhibitor.

18. (canceled)

19. The method of claim 17, wherein the RIPK2 inhibitor is selective over RIP Kinase 1 and/or RIP Kinase 3.

20. A method of treating Alzheimer's disease in a subject in need thereof, the method comprising administering to the subject a therapeutically effective amount of a RIPK2 inhibitor.

21. (canceled)

22. The method of claim 20, wherein the RIPK2 inhibitor is selective over RIP Kinase 1 and/or 3.

23. The method of claim 1, wherein the RIPK2 inhibitor is gefitinib, sorafenib, regorafenib, ponatinib, SB203580, OD36 (6-Chloro-10,11,14,17-tetrahydro-13H-1,16-etheno-4,8-metheno-1H-pyrazolo[3,4-g][1,14,4,6]dioxadiazacyclohexadecine), OD38 ([4,5,8,9-Tetrahydro-7H-2,17-etheno-10,14-metheno-1H-imidazo[1,5-g][1,4,6,7,12,14] oxapentaazacyclohexadecine]), WEHI-435 (N-(2-(4-amino-3-(p-tolyl)-1H-pyrazolo[3,4-d] pyrimidin-1-yl)-2-methylpropyl)isonicotinamide), GSK583 (6-(tert-butylsulfonyl)-N-(5-fluoro-1H-indazol-3-yl)quinolin-4-amine), or a pharmaceutically acceptable salt thereof.

24.-25. (canceled)

26. The method of claim 1, wherein the neurodegenerative disease or disorder comprises: Alzheimer's disease, amyotropic lateral sclerosis (ALS/Lou Gehrig's Disease), Parkinson's disease, diabetic neuropathy, polyglutamine (polyQ) diseases, stroke, Fahr disease, Menke's disease, Wilson's disease, cerebral ischemia, a prion disorder, dementia, corticobasal degeneration, progressive supranuclear palsy, multiple system atrophy, hereditary spastic paraparesis, spinocerebellar atrophies, brain injury or spinal cord injury.

27.-28. (canceled)

29. A method of identifying a therapeutic agent for a neurodegenerative disease or disorder, comprising:
(a) contacting a CNS resident innate immune cell with an abnormally aggregated protein in the presence of a candidate therapeutic agent;
(b) measuring activation of the CNS resident innate immune cell in the presence of the candidate therapeutic agent; and
(c) identifying a therapeutic agent that inhibits activation of the CNS resident innate immune cell compared to a control,
wherein the candidate therapeutic agent is a RIPK2 inhibitor.

30.-31. (canceled)

32. The method of claim 29, wherein the measuring comprises measuring an expression level of NOD2, phosphorylated RIPK2, and/or RIPK2.

33. The method of claim 29, wherein the measuring comprises measuring an expression level of factors C1q, TNFα, and/or IL-1α.

34. The method of claim 29, wherein the measuring comprises measuring an expression level of factors iNOS, Cxcl1, and/or IL-1β; and/or measuring chemotaxis of the CNS resident innate immune cell.

35. The method of claim 29, wherein the therapeutic agent selectively inhibits RIPK2 over RIPK1 and/or RIPK3.

36. The method of claim 29, wherein the therapeutic agent inhibits NOD2-dependent activation of NF-kB.

37. The method of claim 29, wherein the therapeutic agent inhibits amyloid-β aggregates-induced microglial activation, alpha-synuclein aggregates-induced microglial activation and/or A1 astrocyte formation.

38.-39. (canceled)
